(19) United States
(12) Patent Application Publication
(10) Pub. No.: US 2021/0121518 A1
(43) Pub. Date: Apr. 29, 2021

(54) HER2-TARGETED PEPTIDOMIMETICS GRAFTED ONTO MULTICYCLIC PEPTIDE SCAFFOLDS AND METHODS AND USES

(71) Applicant: Board of Supervisors for the University of Louisiana System, Monroe, LA (US)

(72) Inventor: Seetharama D. JOIS, West Monroe, LA (US)

(21) Appl. No.: 16/970,198

(22) PCT Filed: Feb. 15, 2019
(86) PCT No.: PCT/US2019/018228

§ 371 Date: Aug. 14, 2020

Related U.S. Application Data
(60) Provisional application No. 62/631,472, filed on Feb. 15, 2018.

(51) Int. Cl. A61K 38/12; A61K 45/06

(52) U.S. Cl. CPC A61K 38/12; A61K 45/06

(57) ABSTRACT

A method of treating cancer in a mammalian patient comprising administering a pharmaceutical composition to the patient including a pharmacologically effective amount of a first therapeutic, wherein the first therapeutic comprises one or more HER2-targeted peptide functional groups grafted onto a cyclic scaffold, or a pharmaceutically acceptable salt, solvate, ester, amide, clathrate, stereoisomer, enantiomer, prodrug or analogs thereof. In a further embodiment, the one or more HER2-targeted peptide functional groups grafted onto a cyclic scaffold contains at least one 3-amino 3(1-napthyl) propionic acid bound to the scaffold.

### CROSS REFERENCE TO RELATED APPLICATIONS/PRIORITY

The present invention claims priority to U.S. Provisional Patent Application No. 62/631,472 filed Feb. 15, 2018, which is incorporated by reference into the present disclosure as if fully restated herein. Any conflict between the incorporated material and the specific teachings of this disclosure shall be resolved in favor of the latter. Likewise, any conflict between an art-understood definition of a word or phrase and a definition of the word or phrase as specifically taught in this disclosure shall be resolved in favor of the latter.

### BACKGROUND OF THE INVENTION

Aberrant cell signaling through the HER2 tyrosine kinase receptor is a known driver of cancer progression in multiple tissue types, including lung, ovary, and breast. Targeted therapies using tyrosine kinase inhibitors (TKIs) show considerable promise for cancer therapy, but drug resistance following TKI therapy has become a substantial issue. Therapeutic antibodies have been designed to target HER2 specifically; however, the clinical efficacy of these antibodies has been called into question. For the foregoing reasons, there is a pressing need to find new cancer therapeutics.

### SUMMARY OF THE INVENTION

Wherefore, it is the object of the present invention to overcome the shortcoming associated with the prior art.

The presently claimed invention relates to the design and synthesis of cancer therapeutics and, more specifically, to the method of grafting the functional groups of HER2-targeted peptides onto multicyclic scaffolds to increase peptidomimetic stability and efficacy.

The inventor determined to utilize peptides and peptidomimetics as a therapeutic option for pathological targets, but the general instability of peptides presents a fundamental challenge. The inventor knows that thermally and enzymatically stable cyclic peptides can serve as scaffolds to enhance efficacy and biodistribution of therapeutic peptides. A challenge the inventor had to overcome with HER2-targeted peptides is that design and development of peptides with improved stability are needed if peptidomimetics are to be used successfully in the clinic. This report disclosed a method of creating peptidomimetics that are designed to target the HER2 receptor.

The designed peptides bind the HER2 receptor and prevent HER2 dimerization with other receptors of the EGFR family. Inhibiting HER2 protein-protein interactions (PPIs) prevents HER2 dimerization-mediated cell signaling, an event that is known to promote cancer progression. By grafting the functional groups of the peptides onto a cyclic scaffold, the thermal and enzymatic stability of the grafted peptide is improved over the free peptide. In addition, the grafted peptide exhibits potent antiproliferative effects against cancer cells.

The invention relates to novel chemicals and methods of treating cancers, comprising administering to a cancer patient a HER2-targeted peptide functional groups grafted onto a cyclic scaffold (“H2TPFGGoCS”). An additional embodiment includes wherein one or more of the functional groups of the peptide bind to region IV of the extracellular domain of HER2. An additional embodiment includes wherein the cyclic scaffold is chosen from the group of sunflower trypsin inhibitor (SFTI) and theta-defensins.

An additional embodiment includes wherein the grafted peptide sequence is chosen from the group of:

| | |
|---|---|
| SFTI-G1 | Cyclo(C(S-X)RDR(S-X)CFDSIDF) |
| SFTI-G2 | Cyclo(C(R-X)RPpR(R-X)CFF) |
| SFTI-G3 | Cyclo(C(R-X)RRPR(R-X)CF) |
| SFTI-G4 | Cyclo(C(S-X)RPGR(S-X)CFDSIPPDF) |
| SFTI-G5 | Cyclo(C(S-X)RIPPR(S-X)CFPDDF) |
| SFTI-G6 | Cyclo(C(R-X)RIPPR(R-X)CFPDDF) |
| SFTI-G7 | Cyclo(C(S-X)RIHyPHyPR(S-X)CFPDDF) |
| SFTI-G8 | Cyclo(C-(S-X)RI-HyPHyP(S-X)CFPDDF) |
| SFTI-G9 | Cyclo(C(S-X)RIHyPHyPR(S-X)CFpddf) |
| SFTI-G10 | Cyclo(C(S-X)RI-DBF-R(S-X)CFPDDF) |
| SFTI-G11 | Cyclo(C(S-X)RI-HyDBF-R(S-X)CFPDDF) |
| | and |
| SFTI-G12 | Cyclo(C(S-X)-K-RIPPR(S-X)CFPDDF), |

where R-X or S-X is the chirality at the beta amino acid; X is 3-amino 3(1-napthyl) propionic acid, a beta amino acid; in SFTI-G12, K is lysine amino acid, the side chain of K can be attached with fluorescent labels, K can be modified with azide group and attached to a fluorescent label. Single letter code is used for amino acid representation. Capital letters refer to L amino acid, small letter refers to D amino acid (example: P is L proline, p is D proline and HyP is L hydroxyl proline, Hyp is D hydroxyl proline). Disulfide bond between the cysteines is shown in underline. Cyclo indicates a cyclic compound scaffold. DBF is dibenzofuran. HyDBF is hydroxyl dibenzofuran.

An additional embodiment includes wherein the peptide sequence freely changes conformations or is locked into beta conformation. An additional embodiment includes wherein the peptide sequence is locked into beta conformation through incorporation of 1,4 substituted triazole or substitution of Pro-Pro with dibenzofuran.

An additional embodiment includes wherein the cancer is chosen from the group of lung, breast, ovary, bladder, salivary gland, pancreas, and endometrium. An additional embodiment includes wherein the cancer is HER2-positive. An additional embodiment includes wherein HER2 is wild-type or a mutated variant. An additional embodiment includes combining with an effective amount of one or more chemotherapeutic agents from the group of cisplatin, paclitaxel, and docetaxel. An additional embodiment includes combining with an effective amount of one or more tyrosine kinase inhibitors from the group of erlotinib, gefitinib, and lapatinib. An additional embodiment includes wherein the administration route is oral, intravenous, or inhalation. An additional embodiment includes wherein the peptide is administered with pharmaceutically acceptable excipients

An additional embodiment, wherein the peptidomimetics can be used to treat human or canine cancers.

The invention further relates to kits and methods of visualizing HER2+ cancers using a peptide consisting of the sequence Cyclo(C-(S-X)-KRI-PPR-(S-X)-CFPDDF), wherein K is a lysine amino acid side chain that can be conjugated to any fluorescent label and X is amino naphthyl propionic acid (Anapa).

The present invention relates to pharmaceutical compositions of a therapeutic (e.g., an H2TPFGGoCS), or a pharmaceutically acceptable salt, solvate, ester, amide, clathrate, stereoisomer, enantiomer, prodrug or analogs thereof, and use of these compositions for the treatment of a cancer, including lung, breast, ovary, bladder, salivary gland, pancreas, and endometrium cancers.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In some embodiments, the condition is a cancer.

In certain embodiments, the cancer is mild to moderate cancer.

In further embodiments, the cancer is moderate to severe cancer.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

In some embodiments, the pharmaceutical composition is administered concurrently with one or more additional therapeutic agents for the treatment or prevention of the cancer.

In some embodiments, the therapeutic, or a pharmaceutically acceptable salt, solvate, or prodrug thereof, is administered as a pharmaceutical composition that further includes a pharmaceutically acceptable excipient.

In some embodiments, administration of the pharmaceutical composition to a human results in a peak plasma concentration of the therapeutic between 0.05 μM-10 μM (e.g., between 0.05 μM-5 μM).

In some embodiments, the peak plasma concentration of the therapeutic is maintained for up to 14 hours. In other embodiments, the peak plasma concentration of the therapeutic is maintained for up to 1 hour.

In other embodiments, the therapeutic is administered at a dose that is between 0.05 mg-5 mg/kg weight of the human.

In certain embodiments, the pharmaceutical composition is formulated for oral administration.

In other embodiments, the pharmaceutical composition is formulated for extended release.

In still other embodiments, the pharmaceutical composition is formulated for immediate release.

As used herein, the term “delayed release” includes a pharmaceutical preparation, e.g., an orally administered formulation, which passes through the stomach substantially intact and dissolves in the small and/or large intestine (e.g., the colon). In some embodiments, delayed release of the active agent (e.g., a therapeutic as described herein) results from the use of an enteric coating of an oral medication (e.g., an oral dosage form).

The term an “effective amount” of an agent, as used herein, is that amount sufficient to effect beneficial or desired results, such as clinical results, and, as such, an “effective amount” depends upon the context in which it is being applied.

The terms “extended release” or “sustained release” interchangeably include a drug formulation that provides for gradual release of a drug over an extended period of time, e.g., 6-12 hours or more, compared to an immediate release formulation of the same drug. Preferably, although not necessarily, results in substantially constant blood levels of a drug over an extended time period that are within therapeutic levels and fall within a peak plasma concentration range that is between, for example, 0.05-10 μM, 0.1-10 μM, 0.1-5.0 μM, or 0.1-1 μM.

As used herein, the terms “formulated for enteric release” and “enteric formulation” include pharmaceutical compositions, e.g., oral dosage forms, for oral administration able to provide protection from dissolution in the high acid (low pH) environment of the stomach. Enteric formulations can be obtained by, for example, incorporating into the pharmaceutical composition a polymer resistant to dissolution in gastric juices. In some embodiments, the polymers have an optimum pH for dissolution in the range of approx. 5.0 to 7.0 (“pH sensitive polymers”). Exemplary polymers include methacrylate acid copolymers that are known by the trade name Eudragit® (e.g., Eudragit® L100, Eudragit® S100, Eudragit® L-30D, Eudragit® FS 30D, and Eudragit® L100-55), cellulose acetate phthalate, cellulose acetate trimellitiate, polyvinyl acetate phthalate (e.g., Coateric®), hydroxyethylcellulose phthalate, hydroxypropyl methylcellulose phthalate, or shellac, or an aqueous dispersion thereof. Aqueous dispersions of these polymers include dispersions of cellulose acetate phthalate (Aquateric®) or shellac (e.g., MarCoat 125 and 125N). An enteric formulation reduces the percentage of the administered dose released into the stomach by at least 50%, 60%, 70%, 80%, 90%, 95%, or even 98% in comparison to an immediate release formulation. Where such a polymer coats a tablet or capsule, this coat is also referred to as an “enteric coating.”

The term “immediate release” includes where the agent (e.g., therapeutic), as formulated in a unit dosage form, has a dissolution release profile under in vitro conditions in which at least 55%, 65%, 75%, 85%, or 95% of the agent is released within the first two hours of administration to, e.g., a human. Desirably, the agent formulated in a unit dosage has a dissolution release profile under in vitro conditions in which at least 50%, 65%, 75%, 85%, 90%, or 95% of the agent is released within the first 30 minutes, 45 minutes, or 60 minutes of administration.

The term “pharmaceutical composition,” as used herein, includes a composition containing a compound described herein (e.g., H2TPFGGoCS), or any pharmaceutically acceptable salt, solvate, or prodrug thereof), formulated with a pharmaceutically acceptable excipient, and typically manufactured or sold with the approval of a governmental regulatory agency as part of a therapeutic regimen for the treatment of disease in a mammal.

Pharmaceutical compositions can be formulated, for example, for oral administration in unit dosage form (e.g., a tablet, capsule, caplet, gelcap, or syrup); for topical administration (e.g., as a cream, gel, lotion, or ointment); for intravenous administration (e.g., as a sterile solution free of particulate emboli and in a solvent system suitable for intravenous use); or in any other formulation described herein.

A “pharmaceutically acceptable excipient,” as used herein, includes any ingredient other than the compounds described herein (for example, a vehicle capable of suspending or dissolving the active compound) and having the properties of being nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, or waters of hydration. Exemplary excipients include, but are not limited to: butylated hydroxytoluene (BHT), calcium carbonate, calcium phosphate (dibasic), calcium stearate, croscarmellose, cross-linked polyvinyl pyrrolidone, citric acid, crospovidone, cysteine, ethylcellulose, gelatin, hydroxypropyl cellulose, hydroxypropyl methylcellulose, lactose, magnesium stearate, maltitol, maltose, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, stearic acid, sucrose, talc, titanium dioxide, vitamin A, vitamin E, vitamin C, and xylitol.

The term “pharmaceutically acceptable prodrugs” as used herein, includes those prodrugs of the compounds of the present invention which are, within the scope of sound medical judgment, suitable for use in contact with the tissues of humans and animals with undue toxicity, irritation, allergic response, and the like, commensurate with a reasonable benefit/risk ratio, and effective for their intended use, as well as the zwitterionic forms, where possible, of the compounds of the invention.

The term “pharmaceutically acceptable salt,” as use herein, includes those salts which are, within the scope of sound medical judgment, suitable for use in contact with the tissues of humans and animals without undue toxicity, irritation, allergic response and the like and are commensurate with a reasonable benefit/risk ratio. Pharmaceutically acceptable salts are well known in the art. The salts can be prepared in situ during the final isolation and purification of the compounds of the invention or separately by reacting the free base group with a suitable organic or inorganic acid. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

The terms “pharmaceutically acceptable solvate” or “solvate,” as used herein, includes a compound of the invention wherein molecules of a suitable solvent are incorporated in the crystal lattice. A suitable solvent is physiologically tolerable at the administered dose. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

The term “prevent,” as used herein, includes prophylactic treatment or treatment that prevents one or more symptoms or conditions of a disease, disorder, or conditions described herein (e.g., a cancer). Treatment can be initiated, for example, prior to (“pre-exposure prophylaxis”) or following (“post-exposure prophylaxis”) an event that precedes the onset of the disease, disorder, or conditions. Treatment that includes administration of a compound of the invention, or a pharmaceutical composition thereof, can be acute, short-term, or chronic. The doses administered may be varied during the course of preventive treatment.

The term “prodrug,” as used herein, includes compounds which are rapidly transformed in vivo to the parent compound of the above formula. Prodrugs also encompass bioequivalent compounds that, when administered to a human, lead to the in vivo formation of therapeutic. Preferably, prodrugs of the compounds of the present invention are pharmaceutically acceptable.

As used herein, and as well understood in the art, “treatment” includes an approach for obtaining beneficial or desired results, such as clinical results. Beneficial or desired results can include, but are not limited to, alleviation or amelioration of one or more symptoms or conditions; diminishment of extent of disease, disorder, or condition; stabilized (i.e. not worsening) state of disease, disorder, or condition; preventing spread of disease, disorder, or condition; delay or slowing the progress of the disease, disorder, or condition; amelioration or palliation of the disease, disorder, or condition; and remission (whether partial or total), whether detectable or undetectable. “Treatment” can also mean prolonging survival as compared to expected survival if not receiving treatment. As used herein, the terms “treating” and “treatment” can also include delaying the onset of, impeding or reversing the progress of, or alleviating either the disease or condition to which the term applies, or one or more symptoms of such disease or condition.

The term “unit dosage forms” includes physically discrete units suitable as unitary dosages for human subjects and other mammals, each unit containing a predetermined quantity of active material calculated to produce the desired therapeutic effect, in association with any suitable pharmaceutical excipient or excipients.

As used herein, the term “plasma concentration” includes the amount of therapeutic present in the plasma of a treated subject (e.g., as measured in a rabbit using an assay described below or in a human).

As is described further below, preferably only the functional groups of the peptides are grafted onto the scaffold. Grafting the functional groups onto a scaffold leads to increased thermal and enzymatic stability versus the peptide alone, and introduction of a hydroxyproline increased water solubility. The disclosed compound works by, among other things, binding HER2, thereby preventing HER2 from interacting with other cancer promoting receptors. Combining the grafted peptide with erlotinib resulted in surprising synergistic result (antiproliferation in EGFR mutated lung cancer cell lines).

The presently claimed invention is related to compositions and methods of treating cancer in a mammalian patient comprising administering a pharmaceutical composition to the patient including a pharmacologically effective amount of a first therapeutic, wherein the first therapeutic comprises one or more HER2-targeted peptide functional groups grafted onto a cyclic scaffold, or a pharmaceutically acceptable salt, solvate, ester, amide, clathrate, stereoisomer, enantiomer, prodrug or analogs thereof. According to a further embodiment one or more of the functional groups of the peptide bind to region IV of an extracellular domain of HER2. According to a further embodiment the scaffold is one of a sunflower trypsin inhibitor and a theta-defensins. According to a further embodiment the one or more HER2-targeted peptide functional groups grafted onto a cyclic scaffold contains at least one 3-amino 3(1-napthyl) propionic acid bound to the scaffold. According to a further embodiment the one or more HER2-targeted peptide functional groups grafted onto a cyclic scaffold contains at least two 3-amino 3(1-napthyl) propionic acids bound to the scaffold. According to a further embodiment two of the at least two 3-amino 3(1-napthyl) propionic acids have one of the same or different chirality. According to a further embodiment two of the at least two 3-amino 3(1-napthyl) propionic acids are bonded to each other by between 2 and 7 amino acids. According to a further embodiment a sequence of the grafted peptide is one of Cyclo(C(S-X)RDR(S-X)CFDSIDF); Cyclo(C(R-X)RPpR(R-X)CFF); Cyclo(C(R-X)RRPR(R-X)CF); Cyclo(C(S-X)RPGR (S-X)CFDSIPPDF); Cyclo(C(S-X)RIPPR(S-X)CFPDDF); Cyclo(C(R-X)RIPPR(R-X)CFPDDF); Cyclo(C(S-X)RIHyPHyPR(S-X)CFPDDF); (C-(S-X)RI-HyPHyP(S-X)CFPDDF); Cyclo(C(S-X)RIHyPHyPR(S-X)CFpddf); Cyclo(C(S-X)RI-DBF-R(S-X)CFPDDF); Cyclo(C(S-X)RI-HyDBF-R(S-X)CFPDDF); Cyclo(C(S-X)-K-RIPPR(S-X)CFPDDF); where “R-X” and “S-X” indicate the chirality at a beta amino acid; “X” is a beta amino acid 3-amino 3(1-napthyl) propionic acid; “K” is lysine amino acid; single letter code is used for amino acid representation, capital letters refer to L amino acid, lower case letters refers to D amino acid, “Hy” before an amino acid indicates a hydroxyl form of the following amino acid, a disulfide bond between cysteines is shown by underline, “Cyclo” indicates a cyclic compound scaffold; “DBF” is dibenzofuran, and “HyDBF” is hydroxyl dibenzofuran. According to a further embodiment one of a side chain of K is attached with a fluorescent labels and K is modified with azide group and attached to a fluorescent label. According to a further embodiment the pharmaceutical composition further comprises a second therapeutic distinct from the first therapeutic. According to a further embodiment the second therapeutic includes one or more chemotherapeutic agents. According to a further embodiment the second therapeutic includes one or more of cisplatin, paclitaxel, and docetaxel, or a pharmaceutically acceptable salt, solvate, ester, amide, clathrate, stereoisomer, enantiomer, prodrug or analogs thereof. According to a further embodiment the second therapeutic includes one or more tyrosine kinase inhibitors. According to a further embodiment the second therapeutic includes one of erlotinib, gefitinib, and lapatinib, or a pharmaceutically acceptable salt, solvate, ester, amide, clathrate, stereoisomer, enantiomer, prodrug or analogs thereof. According to a further embodiment the pharmaceutical composition further comprises a third therapeutic distinct from the first therapeutic and the second therapeutic. According to a further embodiment the third therapeutic includes one or more tyrosine kinase inhibitors. According to a further embodiment the cancer is one of a lung, a breast, an ovary, a bladder, a salivary gland, a pancreas, and an endometrium cancer. According to a further embodiment the cancer is HER2 positive. According to a further embodiment the peptide sequence one of freely changes conformations, is locked into beta conformation through incorporation of 1,4 substituted triazole, is locked into beta conformation through substitution of Pro-Pro with dibenzofuran, the mammal is one of Homo sapiens and Canis lupus, the cancer is HER2+ and the HER2 is one of wild-type a mutated variant, the administration route is one of oral, intravenous, or inhalation, the first therapeutic is administered with pharmaceutically acceptable excipients.

The presently claimed invention further relates to devices and methods of visualizing HER2+ cancers comprising using a peptide consisting of a sequence Cyclo(C-(S-X)-KRI-PPR-(S-X)-CFPDDF), wherein “S-X” indicates a chirality at a beta amino acid, “X” is a beta amino acid 3-amino 3(1-napthyl) propionic acid; “K” is lysine amino acid, single letter code is used for amino acid representation, capital letters refer to L amino acid and lower case letters refers to D amino acid, “Hy” before an amino acid indicates a hydroxyl form of the following amino acid, a disulfide bond between cysteines is shown by underline, “Cyclo” indicates a cyclic compound scaffold, and X is amino naphthyl propionic acid (Anapa).

Various objects, features, aspects, and advantages of the present invention will become more apparent from the following detailed description of preferred embodiments of the invention, along with the accompanying drawings in which like numerals represent like components. The present invention may address one or more of the problems and deficiencies of the current technology discussed above. However, it is contemplated that the invention may prove useful in addressing other problems and deficiencies in a number of technical areas. Therefore the claimed invention should not necessarily be construed as limited to addressing any of the particular problems or deficiencies discussed herein.

### BRIEF DESCRIPTION OF THE DRAWINGS

The accompanying drawings, which are incorporated in and constitute a part of the specification, illustrate various embodiments of the invention, and together with the general description of the invention described above and the detailed description of the drawings described below, serve to illustrate the key features of the invention. The invention will now be described, using the examples, with reference to the accompanying drawings in which:

FIGS. 1A-1C. show a design concept of a H2TPFGGoCS, here a grafted sunflower trypsin inhibitor (SFTI) peptide based on (FIG. 1A) compound 18, shown in FIG. 1D, that binds to domain IV of HER2 ECD and inhibit EGFR:HER2, HER2:HER3 dimerization. FIG. 1B) Sunflower trypsin inhibitor SFTI peptide. FIG. 1C) Grafting of pharmacophore from 18 to SFTI frame work resulting in one variation of the grafted peptide. Single letter amino acid code was used for peptide sequence. Grafted residues are shown in red. X=amino naphthyl propionic acid (Anapa) specifically, 3-amino 3(1-napthyl) propionic acid.

FIG. 1D shows a table of sunflower trypsin inhibitor (SFTI) template based grafted peptides designed based on compound 18. X=Anapa, amino naphthyl propionic acid, specifically, 3-amino 3(1-napthyl) propionic acid. Single letter amino acid code was used for peptide sequence. Small letters refer to D amino acids and capital letters refer to L amino acids. Chirality of Anapa was indicated with R and S configuration in italics. Antiproliferative activity of peptides in cancer cell lines are given as IC₅₀ μM. Disulfide bond between the cysteines is shown in underline.

FIGS. 2A1-2C shows various HER2 binding. FIGS. 2A1 and 2A2 show SFTI-G5 binds to HER2 protein extracellular domain (ECD). Surface plasmon resonance (SPR) analysis of binding of grafted peptide with HER2 ECD protein at different concentrations. Kinetics of association and dissociation is seen with a Kd value 0.487 μM. FIG. 2B) SFTI-G5 binds to HER2 ECD domain IV. SPR analysis of binding of grafted peptide with HER2 ECD domain IV protein at different concentrations. Kinetics of association and dissociation is seen with a Kd value 0.346 μM. FIG. 2C) A control peptide, H₂N—K(3-aminobiphenyl propionic acid)F—OH, does not bind to HER2 protein shown by SPR analysis. There was no significant change in SPR sensorgram upon addition of a control peptide indicating that control peptide does not bind to HER2 protein.

FIG. 3 shows competitive binding of SFTI-G5 and HER2 ECD with EGFR. SPR analysis of binding of SFTI-G5 and HER2 with EGFR. EGFR ECD was immobilized on SPR chip. When HER2 ECD was added to EGFR, SPR signal was increased indicating HER2 ECD binding to EGFR and dimerization of EGFR:HER2. When compound SGTI-G5 was added with increasing concentration with constant concentration of HER2, SPR signal due to HER2 binding was decreased indicating SFTI-G5 binding competitively to HER2 and inhibiting HER2:EGFR dimerization.

FIGS. 4A-4H show SFTI-G5 inhibits HER2:HER3 and EGFR:HER2 dimerization in Calu-3 lung cancer cells as shown by proximity ligation assay (PLA). FIG. 4A) HER2:HER3 dimerization detected by red fluorescence without any peptide treatment. FIG. 4B) Control. FIG. 4C) & FIG. 4D) Inhibition of dimerization by SFTI-G5 at 0.5 and 1 μM, respectively. FIG. 4E) HER2:EGFR dimerization detected by red fluorescence without any peptide treatment. FIG. 4F) Control. FIG. 4G) & FIG. 4H) Inhibition of dimerization by SFTI-G5 at 0.5 and 1 μM, respectively.

FIG. 5 shows quantification of PLA assay Results for Calu-3 and A549 cells (no images shown). Note the decrease in fluorescence signaling in the presence of SFTI-G5 at 0.5 and 1 μM. Thus, SFTI-G5 inhibits HER2:HER3 dimerization. *p<0.05, **P<0.01.

FIG. 6 shows PPI inhibition assessed by PLA on lung tumor tissue. EGFR:HER2 PPI and its inhibition in SFTI-G5 treated mice. HER3:HER2 PPI and its inhibition by SFTI-G5 treated mice. Note the decrease in red fluorescence in treated mice lung cancer tissue due to inhibition of PPI by SFTI-G5. Magnification 40×. Scale bar 20 μm.

FIG. 7 shows SFTI-G5 (1 μM) inhibits phosphorylation of HER2 kinase compared to the control in A549 cells shown by Western blot and a graph of the densitometric ratios of the blots. Lapatinib (2 μM) was used as positive control. Incubation 40 h. *p<0.05 **P<0.01.

FIGS. 8A-8C show the synergistic effect of the disclosed H2TPFGGoCSs with tyrosine kinase inhibitor anti-cancer medications. In this experiment SFTI-G5 was shown to be synergistic with erlotinib, a tyrosine kinase inhibitor (TKI) on: FIG. 8A) EGFR mutated NSCLC cell line NCI-H1975; FIG. 8B) BT-474 cell lines that overexpress HER2; FIG. 8C) Calu-3 lung cancer cell lines that overexpresses HER2. As shown in the isobologram, the point below the diagonal line indicates synergistic effect of SFTI-G5 with erlotinib.

FIGS. 9A-9C show the synergistic effect of the disclosed H2TPFGGoCSs with tyrosine kinase inhibitor anti-cancer medications. In this experiment SFTI-G5 was shown to be synergistic with lapatinib (a TKI) on: FIG. 9A) EGFR mutated NSCLC cell line NCI-H1975; FIG. 9B) BT-474 cell lines that overexpress HER2; FIG. 9C) Calu-3 lung cancer cell lines overexpresses HER2. As shown in the isobologram, the point below the diagonal line indicates synergistic effect of SFTI-G5 with erlotinib.

FIG. 10. Is a table showing the results of a combination index study (another way of representing synergistic effect). Erlotinib was at a constant concentration of 10 μM and concentration of SFTI-G5 was varied from nM concentration to micromolar concentration. Combination index was determined in H1975 lung cancer cell lines (EGFR double mutation). Then SFTI-G5 was at constant concentration of 3 μM and erlotinib concentration was varied from nM to micromolar range. Combination index was calculated. Similar studies with SFTI-G5 and lapatinib was carried out and combination index was calculated. SFTI-G5 exhibited synergistic effect with erlotinib and lapatinib in H1975 lung cancer cell lines. Combination index (CI): <1=synergistic, 1=additive, >1=antagonistic.

FIG. 11 is a graph showing the thermal stability of SFTI-G5 evaluated by CD spectroscopy, which show that there was no significance difference in the CD spectra when temperature of the sample was changed from 25 to 80° C. Overall conformation of the peptide remained the same at all temperatures studied.

FIGS. 12A-12C are three charts showing thermal stability of the disclosed H2TPFGGoCSs using mass spectrometry. MALDI-TOF-MS of the samples of SFTI-G5 at FIG. 12A) 25° C., FIG. 12B) 50° C., and FIG. 12C) 80° C. indicated that the major molecular ion observed was from intact peptide SFTI-G5 suggesting that peptide was thermally stable.

FIG. 13 shows the stability of the disclosed H2TPFGGoCSs in trypsin. For possible oral administration of SFTI-G5, was tested for stability in the GI tract by a low pH resistance assay, and pepsin and trypsin resistance assays. The trypsin inhibition assay was performed with a modified Erlanger's method. The SFTI-G5 was diluted in 100 mM ammonium bicarbonate buffer (pH 7.4) to a concentration of 0.2 mg/mL and trypsin was added to the peptides at a ratio of 1:50 and incubated at 37° C. Samples at different incubation times were analyzed by HPLC. SFTI-G5 was stable in trypsin assay.

FIG. 14 shows the stability of the disclosed SFTis in human serum (in vitro studies). SFTI-G5 (2 mg/mL) was dissolved in human serum (purchased from Innovative Science). At different time points, aliquots of sample were taken and subjected to extraction of compound using cold acetonitrile. Samples were freeze dried and quantified by HPLC method. SFTI-G5 exhibits stability up to 50 h serum. Nearly 60% of sample is intact peptide.

FIG. 15 shows SFTI-G5 binding to human serum albumin (HSA). Surface plasmon resonance (SPR) analysis of binding of grafted peptide with HSA protein at different concentrations. Kinetics of association and dissociation is seen with a Kd value 56.9 μM suggesting the peptide binds HSA protein weakly.

FIG. 16 comprises fifteen photographs of lung cancer development and treatment by SFTI-G5. A549 cells with luciferase were injected to induce lung cancer in mice (Foxn1-nude). One week after injection of cells, mice were monitored for lung cancer development using luciferin imaging. Peptide was administered via IV twice a week after 2 weeks of cell injections. Representative images: In the control group (row 1) lung cancer tumor growth increased in 5 weeks. In animals that were treated with SFTI-G5 (rows 2 and 3) via IV injection (6 mg/kg, 100 μL), tumor growth was reduced compared to the control group as shown by reduction in bioluminescence of tumor week 4 and 5.

FIG. 17 is a chart showing relative luminescence intensity (total photon flux/sec) plotted for 3 animals and a control. During 4th and 5th week, there was a reduction in the luminescence intensity of tumors in SFTI-G5 treated animals compared to control indicating SFTI-G5 reduces the tumor volume in animal model.

FIG. 18A is a plot of relative luminescence intensity (total photon flux/sec) plotted for 4 animals and controls from the lung cancer study, *p<0.05, and FIG. 18B is a plot displaying bodyweight change of mice during the study.

FIGS. 19A-19C are tissue images. FIG. 19A) Tissue sections show normal lung histology with terminal respiratory bronchioles and open alveoli. The alveolar septa are thin and lined by flat epithelium. No tumor or malignant cells are seen. FIG. 19B) Tissue sections show multiple large tumor nests replacing lung parenchyma (10%). The tumor cells show high nuclear and contain rich eosinophilic cytoplasm. FIG. 19C) Tissue sections show normal lung tissue with a few small tumor nests that involves less than 2% of parenchyma. The tumor cells show high nuclear and contain rich eosinophilic cytoplasm.

FIGS. 20A-20B show the evaluation of expression of HER2. FIG. 20A shows lung tissue of mice that developed cancer using A549 cells. FIG. 20B shows lung tissue of mice that were not injected with A549 cells (control). HER2 expression was evaluated using HER2 affibody with fluorescent label FITC. Note the green fluorescence in mice that were injected with A549 cells indicating HER2 expression. In normal mice, HER2 expression was not seen in lung tissue (FIG. 20B).

FIG. 21 is a graph of the stability of the disclosed H2TPFGGoCSs in mice. Mice were injected with SFTI-G5 (6 mg/kg) in 100 uL of PBS. Blood samples were collected at different intervals of time via tail vein. Samples were analyzed by HPLC and mass spectrometry. Relative AUC was plotted with respect to time. Nearly 50% of SFTI-G5 intact peptide was seen in mice up to 12 h.

FIG. 22 is a photograph that shows a comparison of solubility of SFTI-G5, SFTI-G7 and compound 18 in water. 0.5 mg of each compound was added to water in 1 mL. Notice the turbidity of compound 18 indicating solubility of 18 in water <0.5 mg/mL. SFTI-G5 and SFTI-G7 were slightly turbid compared to blank (water only). Note: For compound to be orally available, it should not be highly soluble in water. If a compound is highly soluble in water, it may not pass through intestinal barrier. Compound should be slightly lipophilic to pass through intestinal barrier.

### DETAILED DESCRIPTION OF THE PREFERRED EMBODIMENTS

The presently claimed invention will be understood by reference to the following detailed description, which should be read in conjunction with the appended drawings. It is to be appreciated that the following detailed description of various embodiments is by way of example only and is not meant to limit, in any way, the scope of the present invention. In the summary above, in the following detailed description, in the claims below, and in the accompanying drawings, reference is made to particular features (including method steps) of the present invention. It is to be understood that the disclosure of the invention in this specification includes all possible combinations of such particular features, not just those explicitly described. For example, where a particular feature is disclosed in the context of a particular aspect or embodiment of the invention or a particular claim, that feature can also be used, to the extent possible, in combination with and/or in the context of other particular aspects and embodiments of the invention, and in the invention generally. The term “comprises” and grammatical equivalents thereof are used herein to mean that other components, ingredients, steps, etc. are optionally present. For example, an article “comprising” (or “which comprises”) components A, B, and C can consist of (i.e., contain only) components A, B, and C, or can contain not only components A, B, and C but also one or more other components. Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

The term “at least” followed by a number is used herein to denote the start of a range beginning with that number (which may be a range having an upper limit or no upper limit, depending on the variable being defined). For example “at least 1” means 1 or more than 1. The term “at most” followed by a number is used herein to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%. When, in this specification, a range is given as “(a first number) to (a second number)” or “(a first number)-(a second number),” this means a range whose lower limit is the first number and whose upper limit is the second number. For example, 25 to 100 mm means a range whose lower limit is 25 mm, and whose upper limit is 100 mm. The embodiments set forth the below represent the necessary information to enable those skilled in the art to practice the invention and illustrate the best mode of practicing the invention. In addition, the invention does not require that all the advantageous features and all the advantages need to be incorporated into every embodiment of the invention.

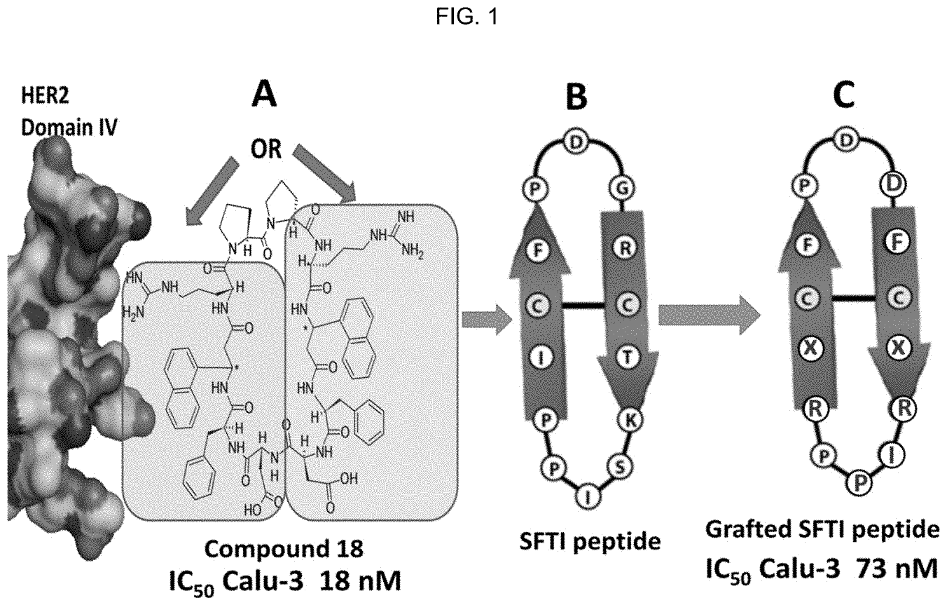

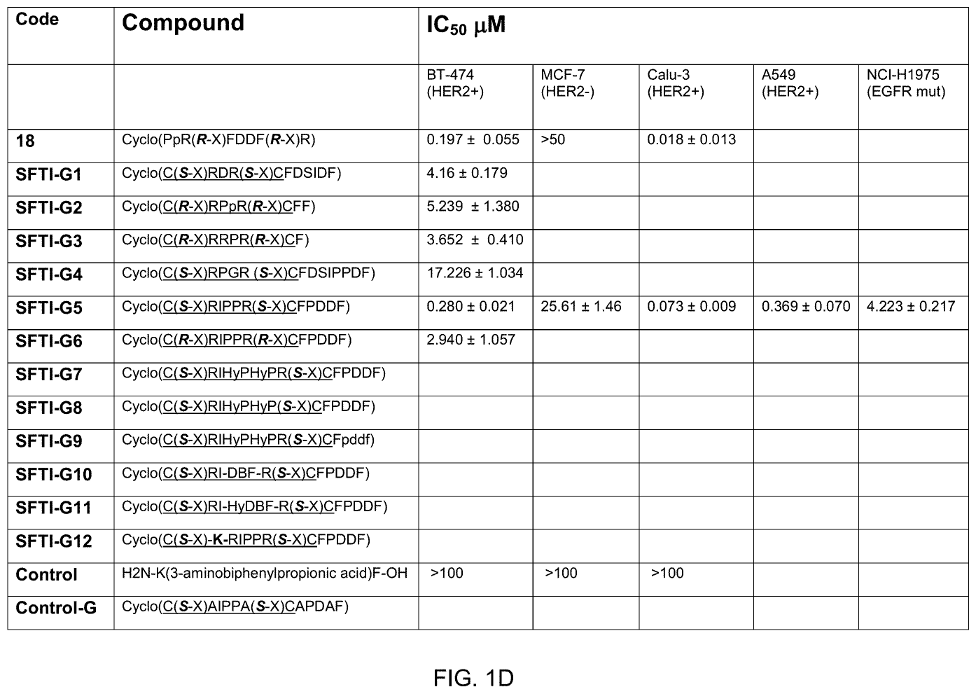

Turning now to FIGS. 1A to 22, a brief description concerning the various components of the present invention will now be briefly discussed. Lung cancer is the second most common type of cancer that occurs in both men and women and is the leading cause of death from cancer in the US. Lung cancer causes more deaths than the next three most common cancers combined (colon, breast, and prostate). Majority of the lung cancer patients are 65 years or older. Thus, with the growing aging population in the United States, this disease will continue to have a major impact on healthy lives and the health care system. The five-year relative survival rate for lung cancer patients is known to be around 18%, a slight increase compared to what it was more than a decade ago, indicating that improvements in lung cancer therapy have been slow. Among the lung cancers, about 85% of lung cancer is histologically classified as non-small-cell lung cancer (NSCLC), a leading cause of cancer deaths worldwide. Traditional chemotherapeutic agents such as cisplatin, paclitaxel, and docetaxel provide standard therapies for lung cancer. However, clinical response is observed in only 30-40% of patients. Chemotherapeutic agents are often met with resistance. However, genotype driven therapy or targeted therapy approaches are promising treatments to treat lung cancer. Studies related to lung cancer have shown a link between human epidermal growth factor receptor-2 (HER2) expression and poor prognosis in patients with NSCLC. Approximately 18-33% of NSCLC tumors show a positive result for HER2 overexpression, suggesting the importance of HER2 in NSCLC. The co-expression of EGFR and HER2 in NSCLC patients was associated with a significantly shortened overall survival rate compared with that in cancer patients whose tumors had high levels of EGFR or HER2 alone.

The inventor is aware that epidermal growth factor receptors (EGFRs) play a major role in lung cancer, as well as in breast and ovarian cancers. Thus, targeting EGFRs can have a significant impact on lung cancer therapy. In addition to human cancers, HER2 is often overexpressed in cancers found in dogs and could serve as a target to treat cancers of canine origin. HER/EGFR system of receptor tyrosine kinases plays an important role in cell growth and differentiation in normal physiology. The receptor system consists of four members: HER1 or EGFR and HER2-4. These proteins have an extracellular domain (ECD), a transmembrane helix, a cytoplasmic kinase domain, and a regulatory region.

Ligand binding to EGFR or HER3 extracellular domains results in a change in the conformation of the proteins, leading to heterodimerization of these proteins and, ultimately, to cell signaling. Among EGFRs, HER2 always exists in the open conformation and is a preferred dimerization partner for other EGFRs. Deregulation of homo- and heterodimerization processes of these receptors or overexpression of receptors leads to different types of cancer and plays a key role in tumor progression, such as tumor growth, increased cell division, vascularization, and metastasis. Possible dimers such as EGFR-HER2, HER2-HER3, and HER2-HER4 have been proposed in the literature. Among these, EGFR-HER2 and HER2-HER3 are present in different types of cancer. Mutation in EGFR seems to play a major role in NSCLC. Because of the limitations of chemotherapy for lung cancer, EGFR-targeted therapy has attracted attention. Targeted therapies are currently approved for these abnormalities and show considerable promise. However, drug resistance has become a substantial issue.

The inventor is aware that heterodimerization of EGFR in the extracellular domain and transphosphorylation of kinase domain results in downstream signaling in pathways such as phosphatidylinositol-3 kinase (PI3K/Akt). HER2 plays an important role in dimerization of receptors and phosphorylation and is important in driving the mitogen-activated protein kinase (MAPK) and PI3K/Akt pathways. Targeting HER2 and inhibiting EGFR:HER2 and HER2:HER3 dimerization will have a significant impact on HER2-overexpressed lung cancer, in particular, NSCLC. In clinical studies, approximately 18-33% of NSCLC tumors showed the 2+/3+ level of HER2 using Herceptest™. Among the heterodimers of EGFR, HER2-HER3 and EGFR-HER2 heterodimers are more abundant in NSCLC. The significance of EGFR and HER2 co-overexpression in NSCLC suggests that targeting both EGFR and HER2 receptors simultaneously may be an advantageous treatment approach.

Extracellular domains of EGFRs homo- and heterodimerize by interacting with domain II and domain IV protein-protein surfaces. Crystal structures of EGFR homodimer suggested that domain IV interactions in the dimer are dominated by hydrophobic residues such as Leu, Trp, and Tyr. One objective of the inventor is in targeting these hot spots with grafted peptides to modulate cell signaling. The inventor is aware that there are “hot spots” on the protein-protein interface area where major contributions to binding free energy are provided. These hot spots of proteins can be targeted with peptides/peptidomimetics or small molecules to modulate PPI.

Peptides have limitations in terms of stability and delivery, and hence the inventor used a novel approach to design grafted peptides using multicyclic stable peptides from sunflower trypsin inhibitor to inhibit EGFR dimerization. The inventor's approach uses a grafted peptide for inhibition of the PPI of EGFR. Multicyclic plant-derived peptides (cyclotides), or mini-proteins that have a cyclic structure with disulfide bonds, are resistant to thermal, chemical, and enzymatic degradation, and are orally bioavailable. Generally, these multicyclic peptides do not present any immunogenicity and are amenable to sequence modification. Although antibodies have been targeted to HER2 and EGFR protein for the treatment of cancer, they have limitations in terms of stability, immunogenicity, and formulation. Therefore, the inventor has designed grafted multicyclic peptides based on a plant peptide sunflower trypsin inhibitor (SFTI) to inhibit EGFR dimerization.

Design of grafted peptides: Peptidomimetics were designed to bind to HER2 protein domain IV and inhibit PPI of HER2:HER3 and EGFR:HER2. Among the compounds designed, compound 18 exhibited antiproliferative activity with IC50 value in the low nanomolar range in HER2 overexpressing cancer cell lines, including lung cancer cell line Calu-3 (FIG. 1D) and, nearly 200 times more selective for HER2 overexpressing cell lines compared to normal breast epithelial cell line MCF-10A (IC₅₀ 40 μM). Details of how compound 18 inhibits EGFR dimerization is reported.

Although compound 18 exhibited PPI inhibition and antiproliferative activity, there were limitations in terms of in vivo stability. One of the main limitations of peptides in vivo has been their short half-life in the circulation, which is caused mainly by proteolytic degradation and/or fast renal clearance. To improve the stability of compound 18, the inventor grafted the important functional groups from compound 18 (FIG. 1A) onto sunflower trypsin inhibitor framework (FIG. 1B) to create a grafted SFTI peptide (FIG. 1C). Sunflower trypsin inhibitor may be used as a template for the design of stable peptides and peptidomimetics. SFTI framework was the simplest (14 amino acids) and smallest with a single disulfide peptide and can be compared to compound 18 in terms of its β-strand structure.

The inventor overlapped the compound 18, and SFTI structure and key residues in compound 18 were grafted on to SFTI sequence without altering the disulfide bond. Resulting SFTI-G1 to G6 peptide structures shown in the table of FIG. 1D. Two control compounds, a linear peptide and a cyclic analog Control-G, were used as controls.

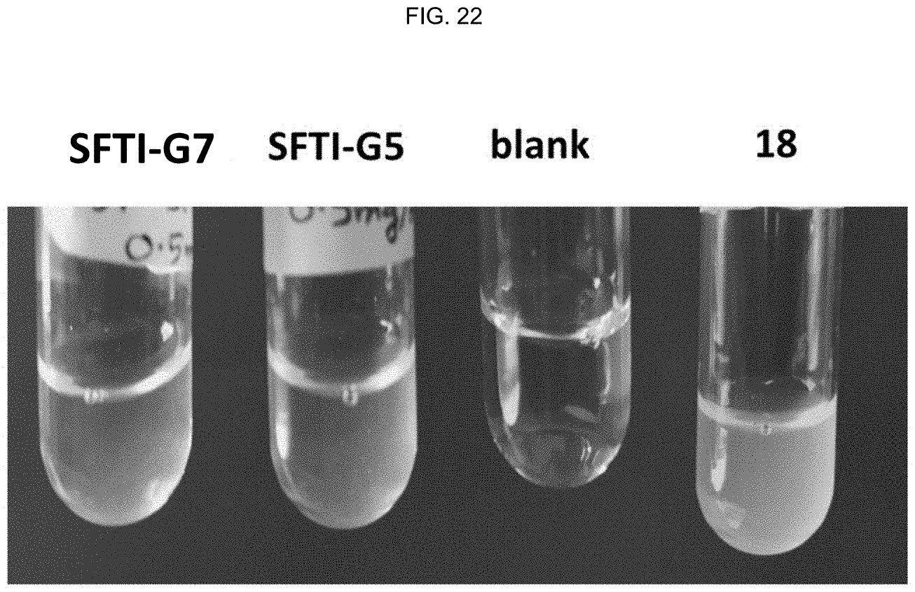

Compounds SFTI-G7, 8 and 9 are analogs of SFTI-G5 that were created to be more water-soluble than compound 18 (FIG. 22, SFTI-G8 and G9 not shown). SFTI-G12 was developed as an imaging agent for HER2+ cancer cells by conjugating with fluorescent labels.

Grafted peptides exhibit antiproliferative activity: Among the designed grafted peptides, SFTI-G5 exhibited the most potent antiproliferative activity with an IC₅₀ value of 280 nM in HER2 overexpressing BT-474 breast cancer cell lines and 73 nM in Calu-3 lung cancer cell lines. Further, it was highly specific for HER2 positive cancer cell lines (FIG. 1, Table 1). The inventor's approach is novel because the molecule the inventor has designed inhibits not only EGFR:HER2 dimerization, but also HER2:HER3 dimerization. The designed peptides target the extracellular domain of the HER2 protein and inhibit HER2:HER3 interaction; hence, the peptides do not have to cross the cellular membrane.

Evaluation of the antiproliferative activity of compounds in HER2-overexpressing cancer cell lines indicated that among the grafted peptides designed, SFTI-G5 exhibited an IC₅₀ value of 280 nM in breast cancer cell lines BT-474 and 73 nM in HER2-overexpressing lung cancer Calu-3 cell lines. On the other hand, in MCF-7 cell lines that do not overexpress the HER2 protein, the IC50 was 25 μM. In normal breast epithelial cell line MCF-10A, compound SFTI-G5 exhibited antiproliferative activity with an IC₅₀ value of 40 μM, nearly 300 times less than its activity against HER2-ve cell lines more than 500 times less in noncancerous breast cell lines compared to a lung cancer cell line (Calu-3). Thus, compound SFTI-G5 is selective for HER2 overexpressing cancer cell lines, in particular HER2 overexpressing lung cancer cell lines.

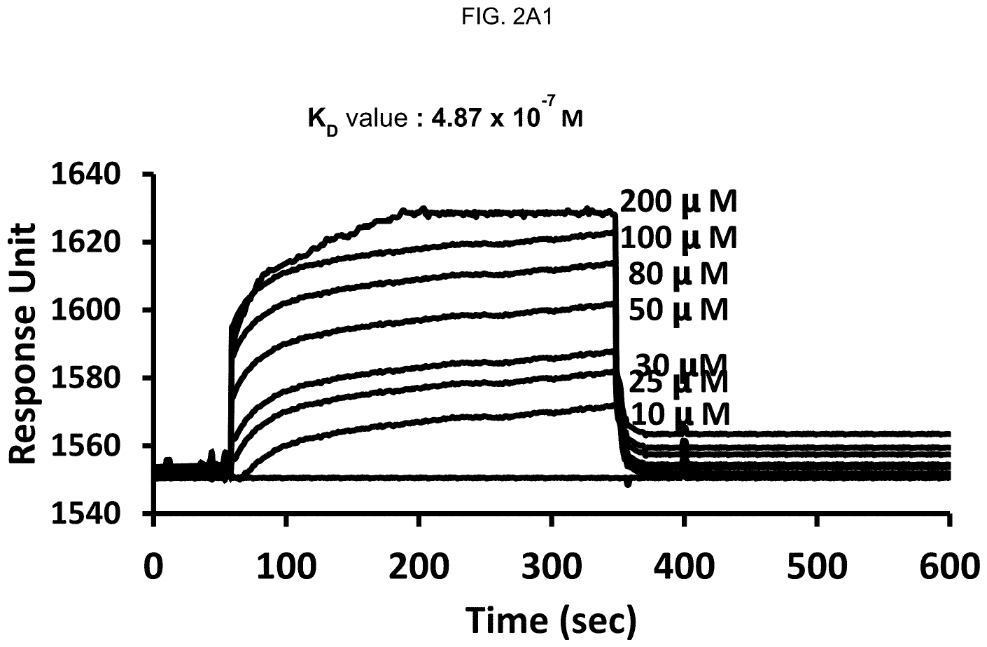

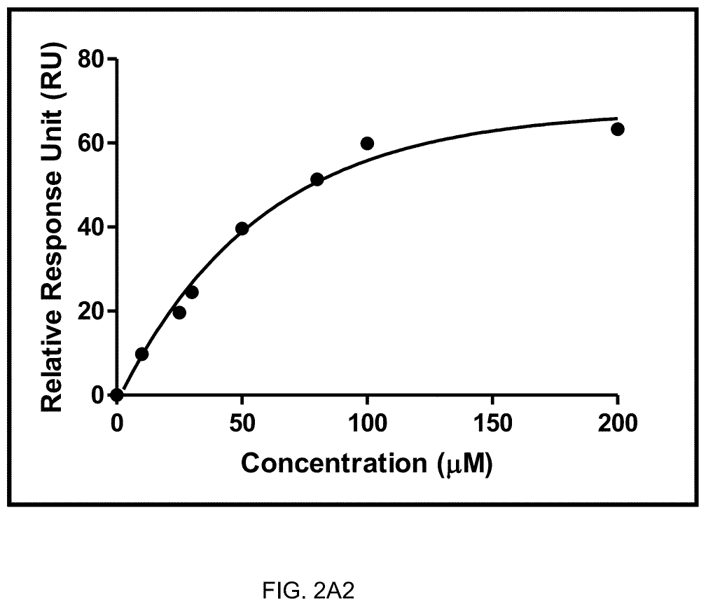

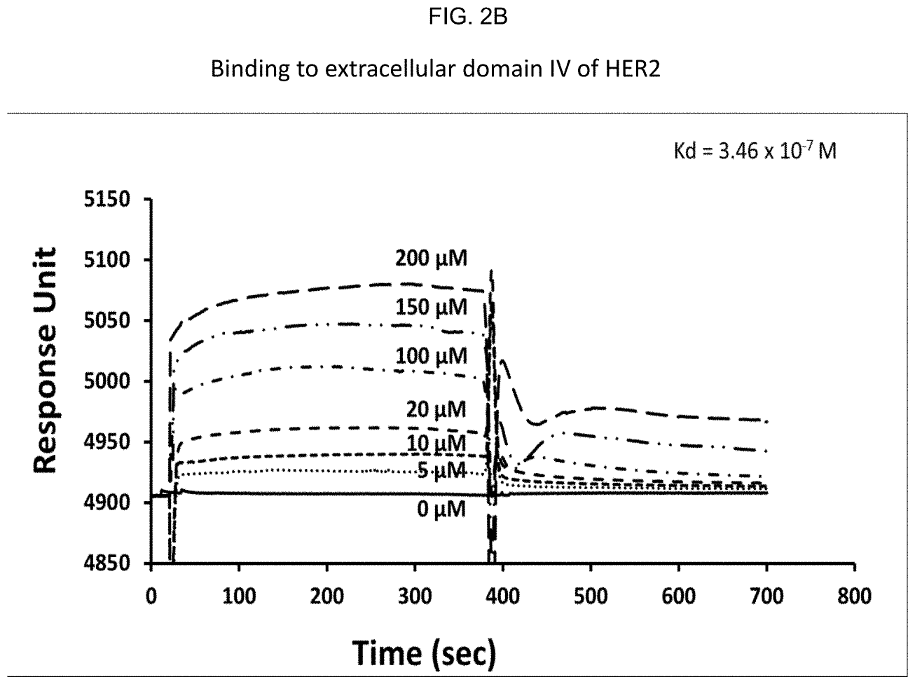

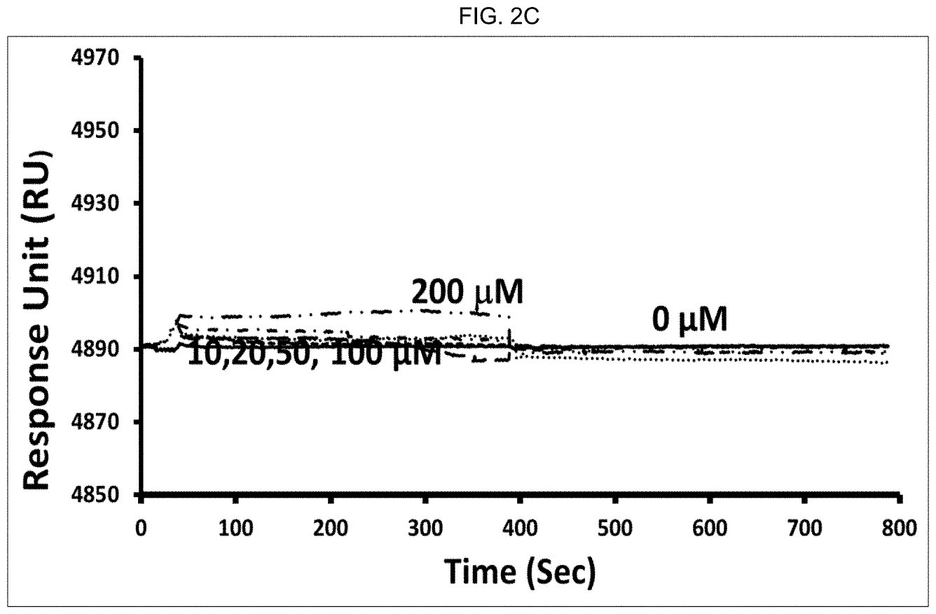

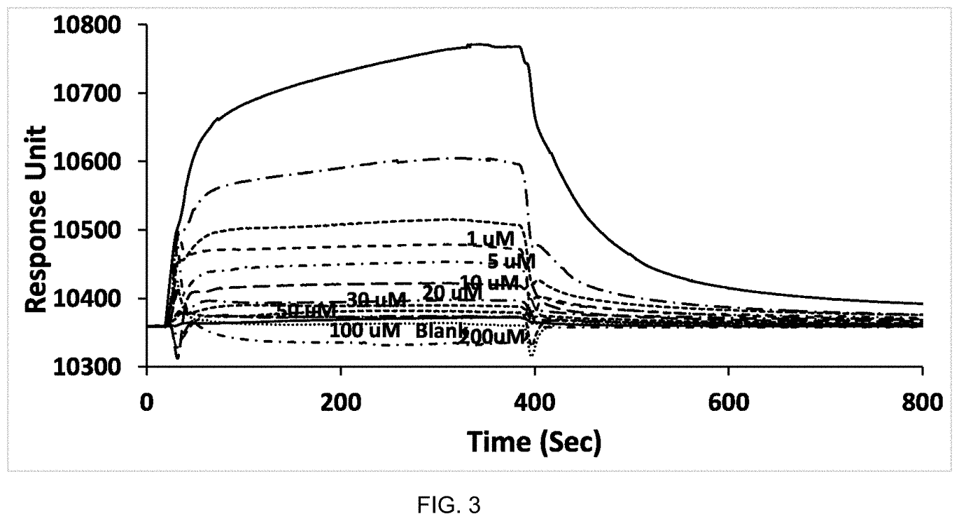

SFTI-G5 binds to domain IV of the HER2 extracellular domain (ECD): The inventor hypothesized that the grafted peptide binds to the HER2 protein, specifically to domain IV of ECD of HER2 and inhibits PPI of EGFR:HER2 and HER2:HER3. This inhibition results in inhibition of signaling for the growth of cancer cells. To evaluate the binding of the grafted peptide to HER2 protein ECD, surface plasmon resonance (SPR) analysis was used. Binding kinetics of SFTI-G5 with HER2 protein immobilized on a CM-5 chip indicated that the peptide binds to the HER2 ECD in a concentration-dependent manner with a Kd value of 0.487 μM (FIG. 2A) and domain IV of the ECD with a Kd value of 0.346 μM (FIG. 2B). The Kd values obtained were consistent with the antiproliferative activity of the peptide in the nM range. When a control peptide was analyzed for binding to a HER2 protein, no binding was observed (FIG. 2C). Competitive binding assays confirm that SFTI-G5 competes with EGFR for binding to the HER2 ECD (FIG. 3), evidencing that SFTI-G5 inhibits HER2:EGFR dimerization.

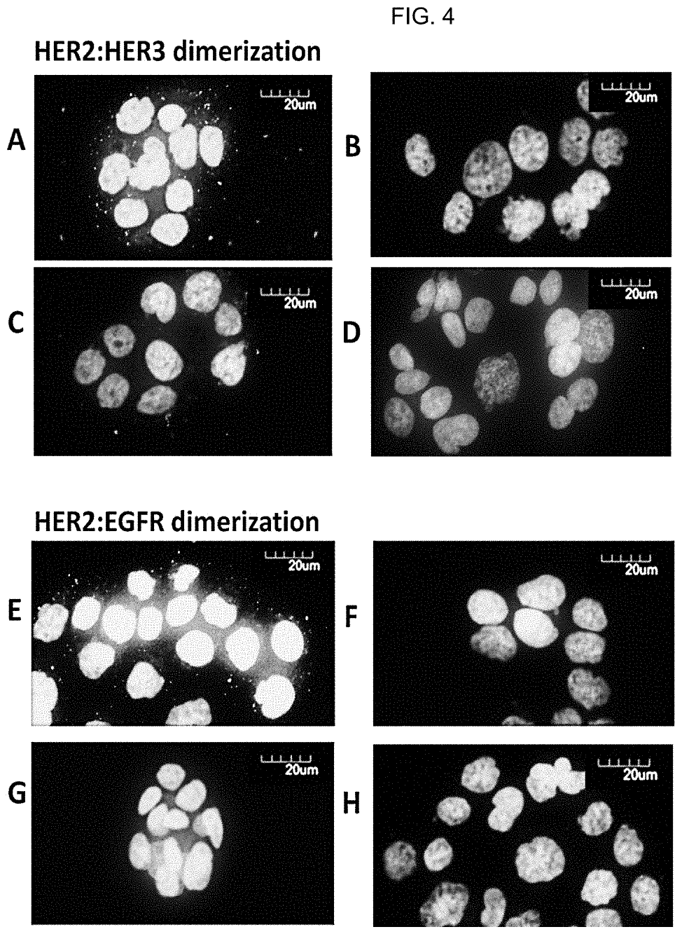

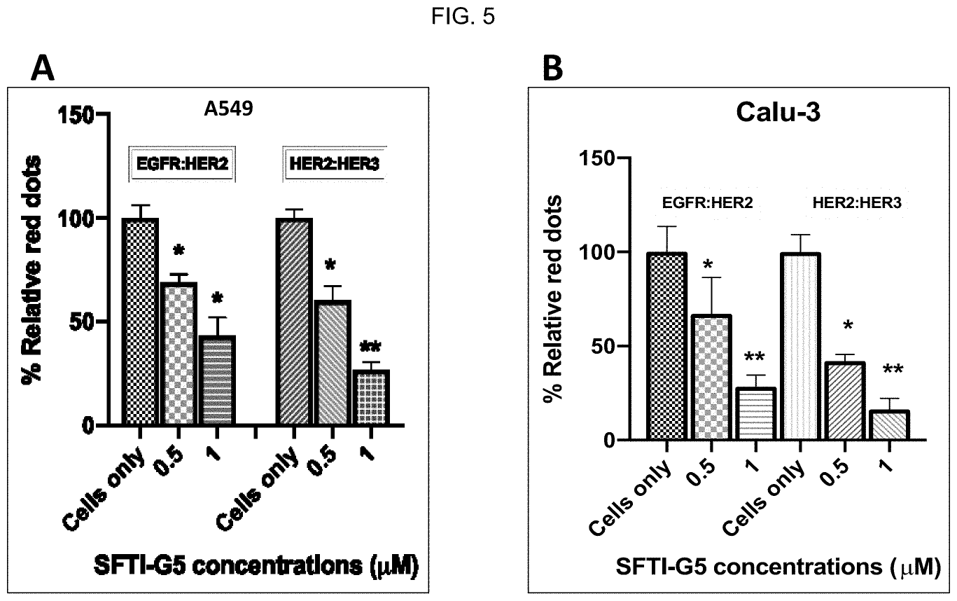

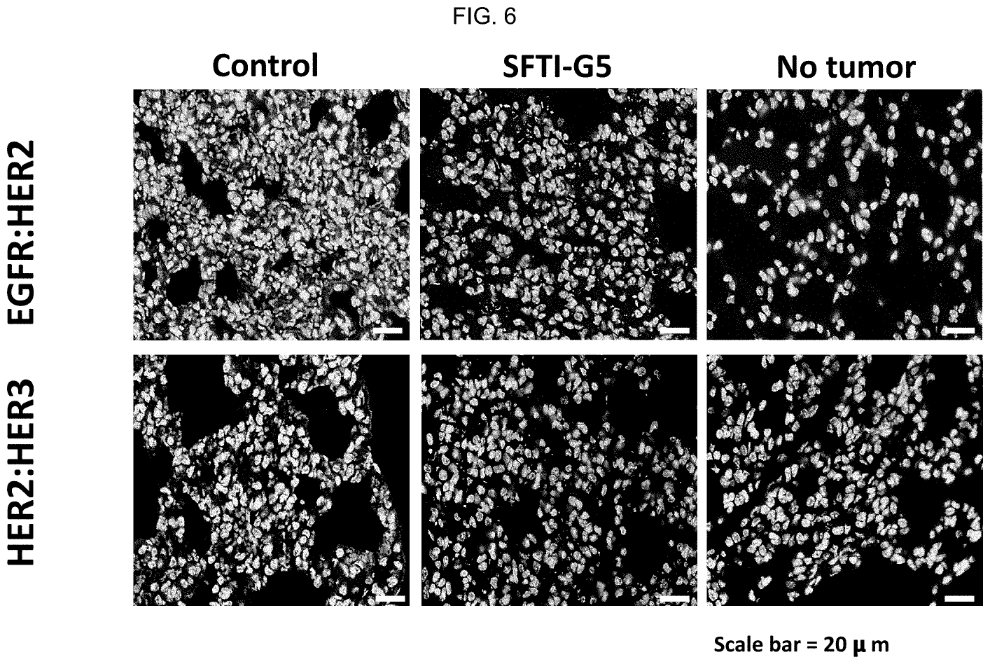

SFTI-G5 inhibits PPI of EGFR:HER2 and HER2:HER3: The ability of grafted peptide SFTI-G5 to inhibit PPI of EGFR was evaluated in HER2 overexpressing lung cancer cell lines Calu-3. Incubation of grafted peptide with Calu-3 cells lines and evaluation of PPI inhibition was carried out using proximity ligation assay (PLA). SFTI-G5 at 0.5 and 1 μM concentration showed inhibition of EGFR:HER2 and HER2:HER3 dimerization (FIGS. 4 and 5B). A549 cells also showed PPI of EGFR:HER2 and HER3:HER2 following treatment with SFTI-G5 (images not shown, quantitation in FIG. 5A). PPI inhibition of EGFR:HER2 and HER2:HER3 was also observed in lung tumor tissue from mice treated with SFTI-G5 (FIG. 6). Thus, the grafted peptide is a dual inhibitor of EGFR dimerization.

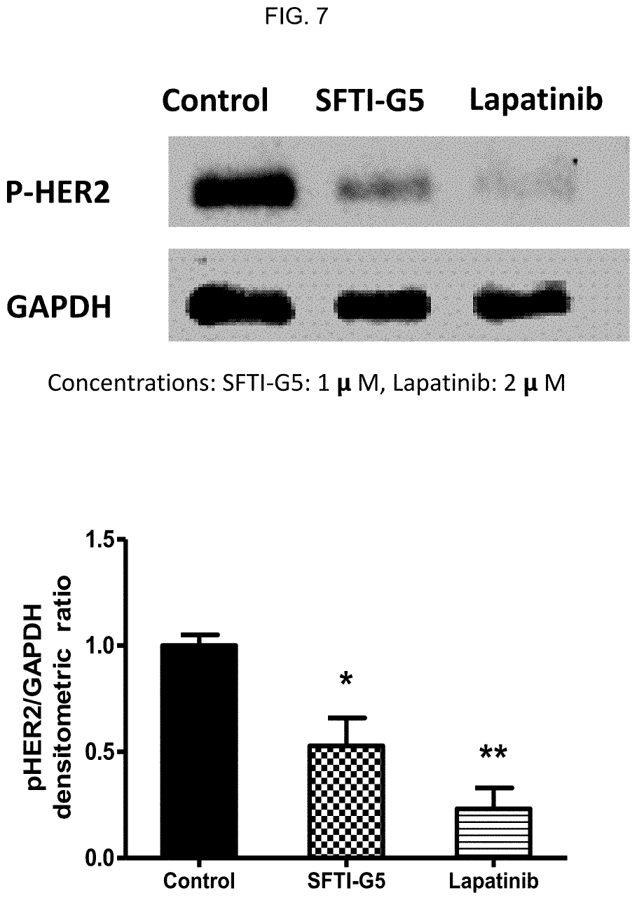

SFTI-G5 inhibits HER2 phosphorylation: Phosphorylation and subsequent activation of HER2 occurs following dimerization of HER2 with another tyrosine kinase receptor. FIGS. 4-6 showed SFTI-G5 blocks PPI of EGFR:HER2 and HER3:HER2, so inhibition of dimerization might also reduce downstream phosphorylation of HER2. Western blot analysis in A549 cells showed SFTI-G5 reduces HER2 phosphorylation (FIG. 7). Lapatinib, a known inhibitor of HER2, was used as a positive control. These data suggest inhibition of HER2 PPI with other EGFR family receptors significantly reduces HER2 activation.

SFTI-G5 exhibits antiproliferative activity in mutated lung cancer cell lines: Drug resistance is common drawback with the use of kinase inhibitors for cancer therapy. Patients receiving first generation kinase inhibitors, such as erlotinib and gefitinib, invariably develop drug resistance, and in nearly 50% of NSCLC cases, the resistance to TKI therapy is due to the presence of T790M mutation. The inventor wanted to evaluate whether compound SFTI-G5 has any effect on the T790 mutation. The inventor therefor designed compounds to bind to the extracellular domain and inhibit the downstream signaling. NCI-H1975 is a cell line that harbors the EGFR L858R/T790M double mutation. Antiproliferative activity of compound SFTI-G5 in NCI-H1975 was 4.22 μM which is much higher (low potency) than in HER2 overexpressing cancer cell lines, while IC₅₀ for erlotinib was 14 μM in NCI-H1975 cell lines (FIG. 1D). Thus, compound SFTI-G5 has better potency than erlotinib in mutated lung cancer cells.

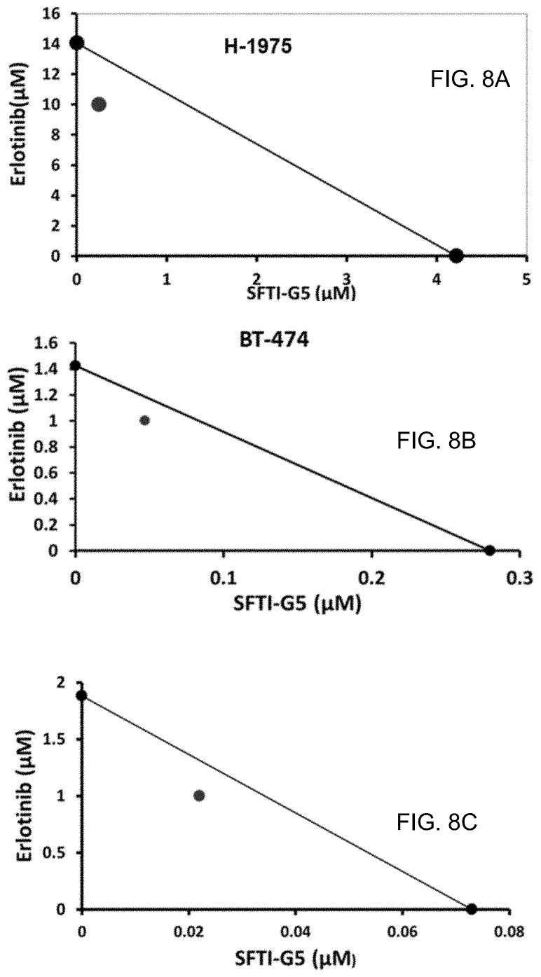

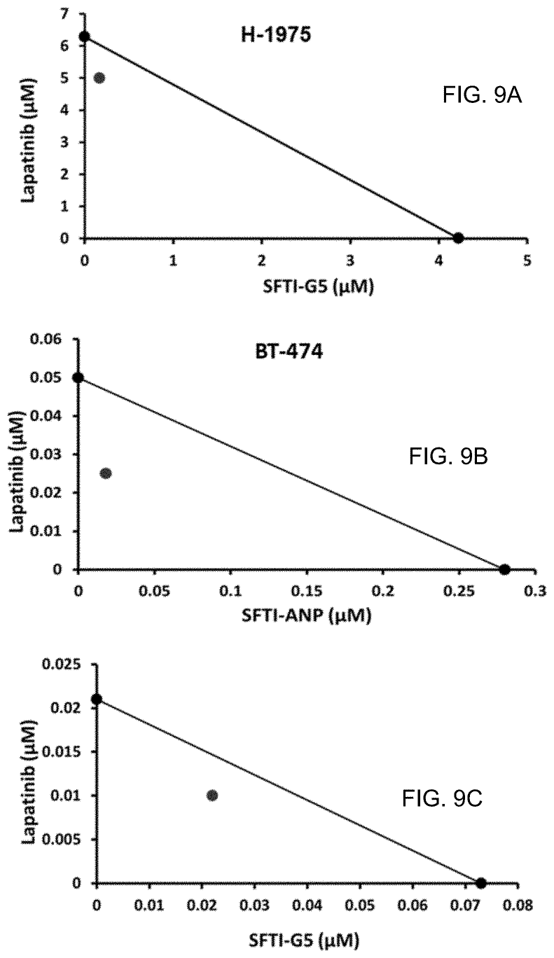

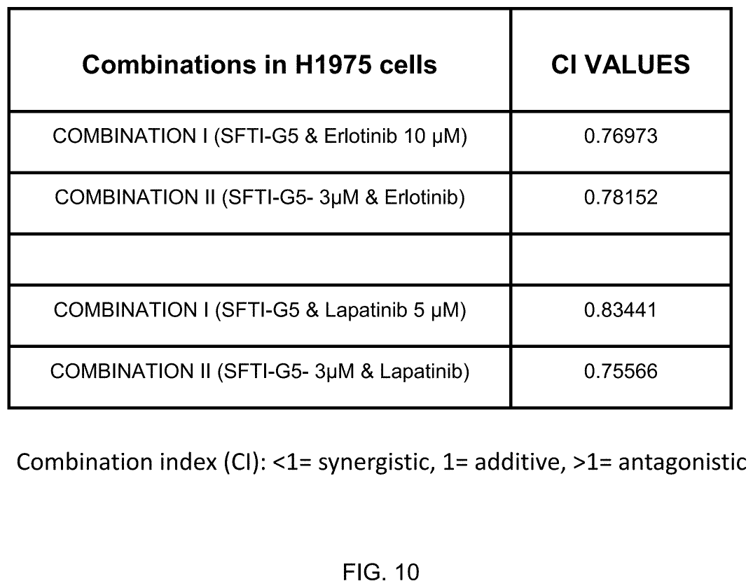

SFTI-G5 exhibits synergistic effect on lung cancer cell lines with erlotinib and lapatinib: The inventor then decided to explore the use of combination therapy as an approach for cancer therapy. To this end, the inventor evaluated whether SFTI-G5 has any synergistic effect with erlotinib or lapatinib in different cancer cell lines, including NCI-H1975 cells. Synergistic effect of SFTI-G5 and erlotinib was evaluated in HER2 overexpressing BT-474, as well as on NCI-H1975 cell lines. Isobolograms were plotted to determine synergistic effect. The inventor's data clearly demonstrated that SFTI-G5+erlotinib and SFTI-G5+lapatinib exhibited synergistic effects in BT-474, Calu-3, and EGFR mutated lung cancer cell lines NCI-H1975 (FIGS. 8 and 9). Combination index (CI) values are another way of conveying synergy, where values <1=synergistic, 1=additive, >1=antagonistic. SFTI-G5 combined with 3 μM or 10 μM erlotinib or lapatinib had CI values <1, suggesting a synergistic effect (FIG. 10). These studies suggest that SFTI-G5 has the potential to be used as a therapeutic agent in lung cancer cell lines in combination with standard of care TKIs.

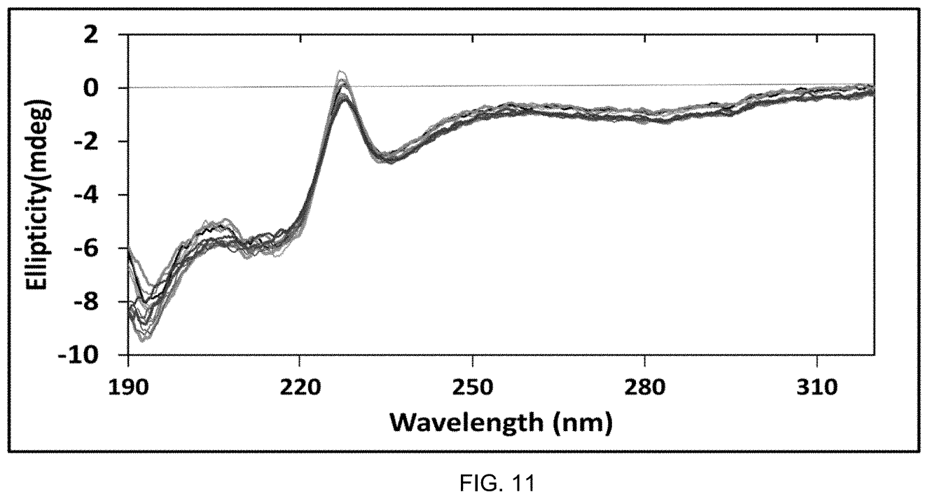

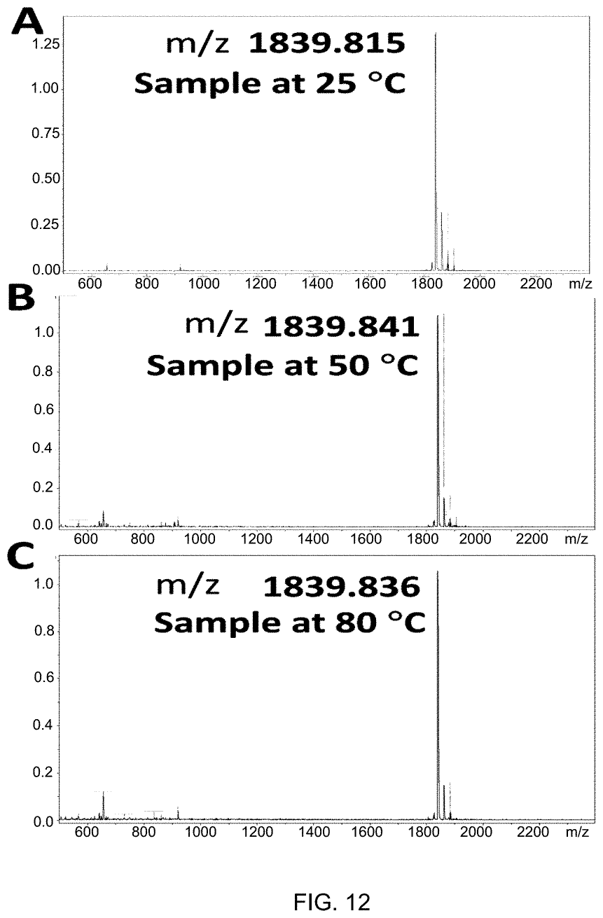

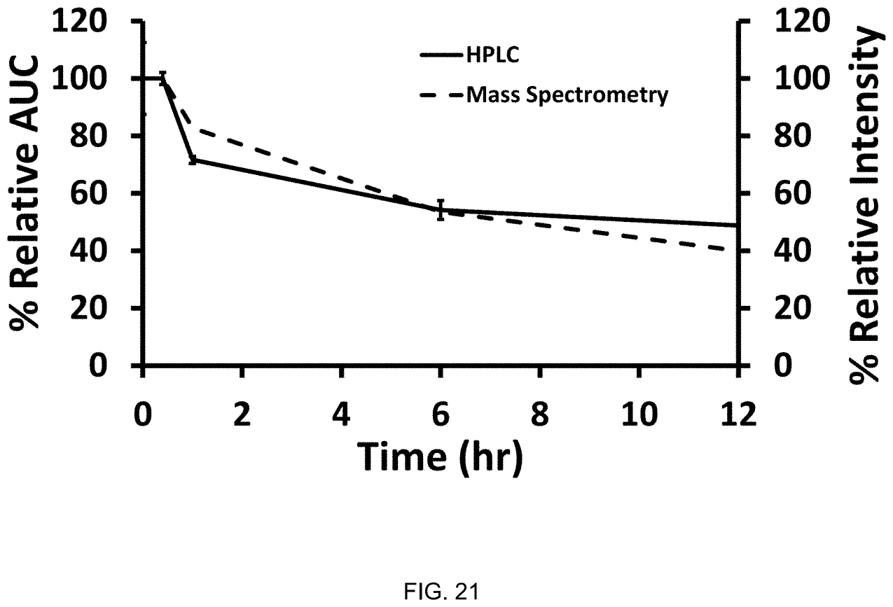

SFTI-G5 exhibits thermal stability: The idea of grafting the peptide using cyclotides or multicyclic peptide, such as SFTI, is to stabilize the peptide framework. To evaluate the thermal stability of SFTI-G5, the inventor monitored the circular dichroism (CD) spectra of SFTI-G5 at different temperatures. CD studies indicated that in solution, the CD spectra of the peptide did not show a significant change in conformation indicating that peptide is stable from 25 C to 85 C (FIG. 11). However, CD spectra of peptides provide the information about the overall average conformation of the peptide in solution. To verify that the peptide was stable even at high temperature, the samples of peptide that were heated to 80 C was analyzed by mass spectrometry. Mass spectrometry analysis suggested that the peptide was stable up to 80 C (FIG. 12). Comparison of the CD spectra of SFTI-G5 (FIG. 11) to the CD spectra of compound 18 (FIG. 21) shows SFTI-G5 is more stable than compound 18. The CD spectra of compound 18 was also verified using mass spectrometry analysis (FIG. 22).

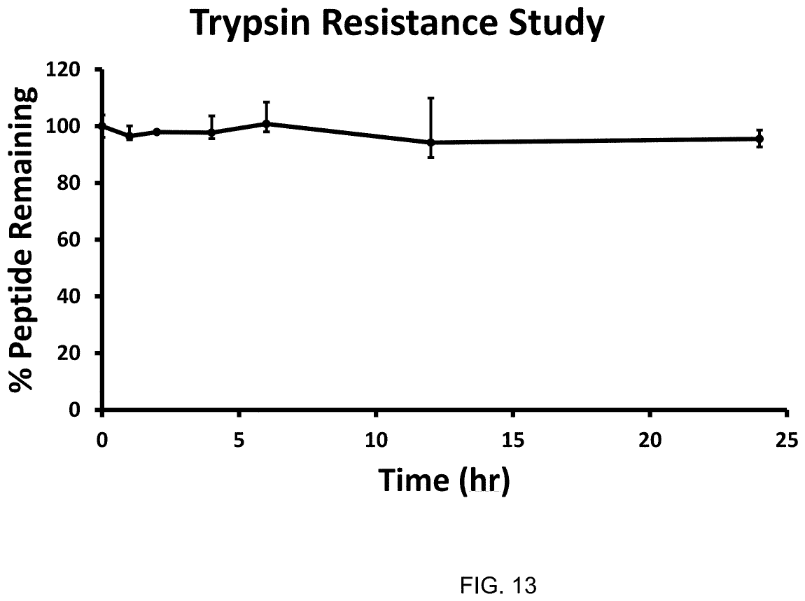

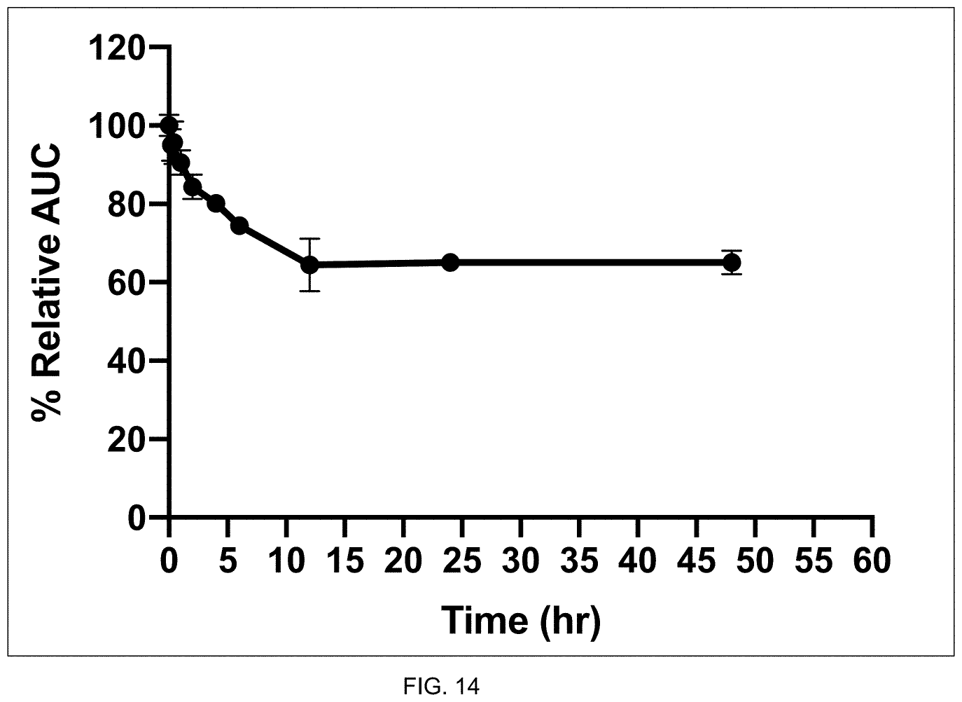

SFTI-G5 exhibits stability in trypsin, human serum and mice: To determine if SFTI-G5 could be used as an orally administered compound, stability in conditions that mimic physiological environments was measured. Stability of SFTI-G5 in trypsin was analyzed as a measure of determining stability in the GI tract. Results showed that SFTI-G5 was stable in 0.5 ug/ul trypsin at pH 7.4 37 C for 24 hours (FIG. 13). Moreover, SFTI-G5 was stable in human serum for up to 50 hours (FIG. 14) and 50% of intact SFTI-G5 in mice was detected 12 hours post-injection (FIG. 21).

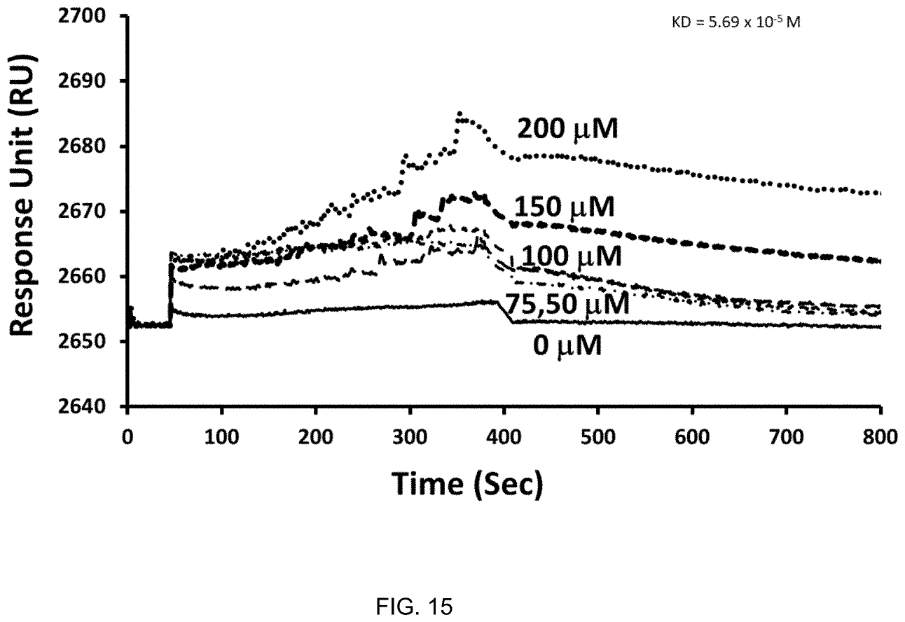

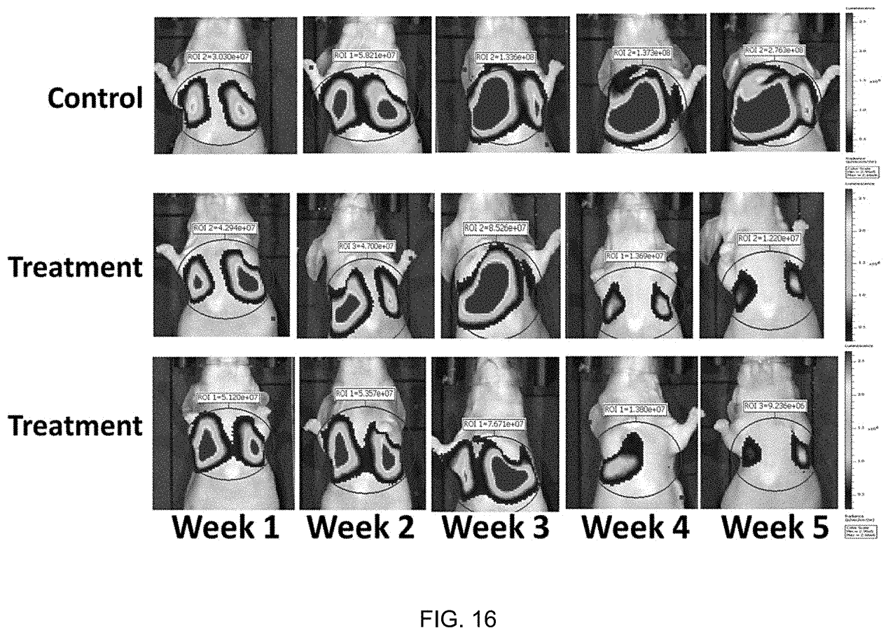

SFTI-G5 binds to serum proteins: Plasma contains various proteins and several functional proteins as carriers, such as human serum albumin (HSA), a1-acid glycoprotein (AGP) and lipoproteins. Among plasma proteins, HSA and AGP play major roles by binding to most drugs. The extent of plasma protein binding of a drug influences the drug's action as well as its distribution and elimination. If a compound is highly bound to plasma proteins, then it results in a low volume of distribution. Extensive plasma protein binding also limits the amount of compound available to be metabolized and hence reduce the clearance of the compound. Complete characterization of the mechanism by which drugs bind to proteins such as HSA and AGP is important for the pharmacokinetic and pharmacodynamic profiles of drugs. Using SPR analysis, the inventor has shown that the grafted peptide binds to human serum albumin (FIG. 15) and the Kd value obtained from the binding kinetics was 56 μM. This binding clearly indicates that the SFTI-G5 does not bind to serum proteins with high affinity and hence serum proteins serve as a carrier for the grafted peptides. This also serves to increase the plasma half-life of the grafted peptide in serum.

SFTI-G5 reduces tumor growth: To evaluate the feasibility of development of lung cancer model and to evaluate the therapeutic potential of the peptide, a pilot study was carried out with a small group of animals (5 animals). Athymic nude mice (Foxn1-nude, 6-7 weeks old) were purchased from Harlan laboratories. Mice were acclimatized for one week in the vivarium. 4.5×10⁶ A549 Red-FLuc NSCLC cells in 150 μL PBS were injected into each mouse via tail vein (IV). Total of four mice were injected with cells. Mice were monitored for 2 weeks. After 1 week, mice were injected with luciferin (200 μL in PBS, IP injection) and imaged under anesthesia using IVIS (Perkin Elmer) instrument. Bioluminescence was measured from each animal. One animal was used as a control. Peptide treatment started after 2 weeks from injection of cells and peptide was prepared in PBS and administered via i.v. with a dose of 6 mg/kg in 100 μL twice a week. Animals were imaged once a week to monitor the progression of lung cancer. Images were quantified using Living Image Software (Perkin Elmer). Bioluminescence signal for each mouse was expressed as total flux of photons/sec. Region of interest tool was used to measure the total photon flux/sec. A graph of bioluminescence for each mouse was plotted with respect to time (each week). After five weeks mice were terminated, and tumor sections and lungs were removed by surgical procedure. Tumor sections were fixed, embedded in parafilm, and cut into 5 μm slices. These were mounted on slides that were stained with HER2 antibody labeled with FITC (Anti-ErbB2 Affibody® (FITC), Abcam Cambridge, Mass.). As a control, lung tissue from normal mice was used. Images were viewed under a fluorescence microscope (Olympus BX63 microscope) at 40× magnification.

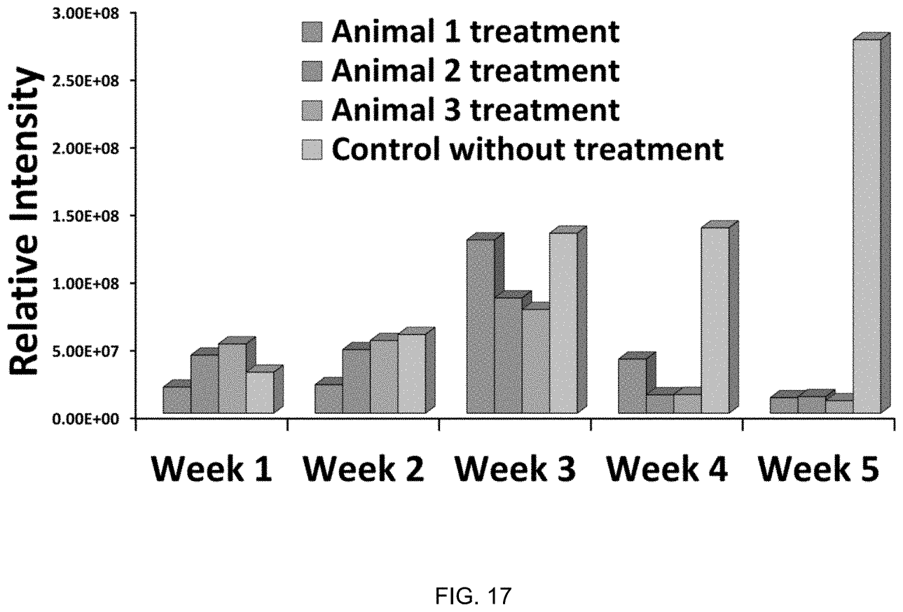

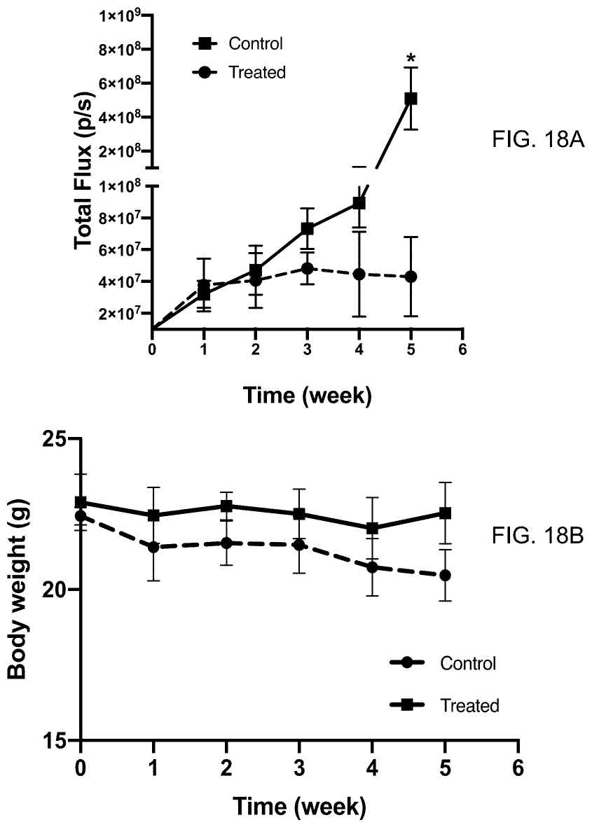

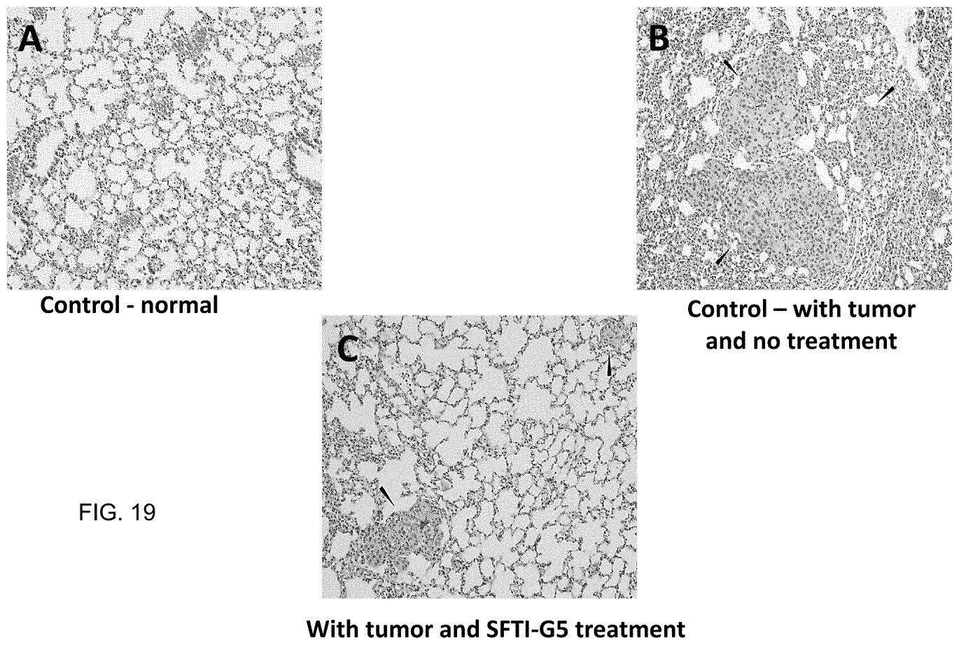

It is established that there is a direct correlation between bioluminescence signal and lung tumor volume. Bioluminescence imaging (FIG. 16) indicates that over 5 weeks, the control animal lung tumors grew while peptide treated animal showed a reduction in tumor size. Quantitative analysis of bioluminescence demonstrates that peptide SFTI-G5 reduced growth of tumors in an animal model of lung cancer (FIGS. 17 and 18A). Histological analysis shows SFTI-G5 reduced pathological features associated with tumor development (FIG. 19). No changes in body weight were observed (FIG. 18B).

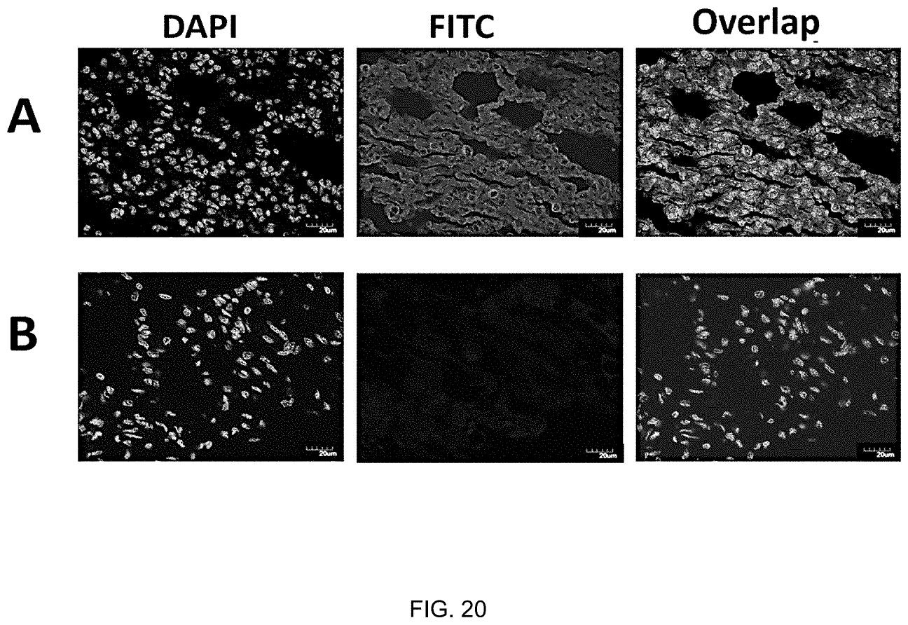

Expression of HER2 in cells and tumor: The inventor used A549-Luc, a non-small cell lung cancer cell line known to be KRAS mutant and EGFR wild-type. A549 is known to overexpress HER2 protein but relatively less compared to Calu-3 cells. The inventor wanted to evaluate the expression of HER2 in lung tumor tissue. Tumor sections that were fixed were embedded in parafilm and cut into 5 μm slices. These were mounted on the slides and slides were used for evaluation of HER2 overexpression using HER2 affibody labeled with FITC. As a control, lung tissue from normal mice was used. A549 cells used for lung cancer development in mice showed HER2 overexpression (FIG. 20A) compared to normal lungs (FIG. 20B).

Pharmaceutical Compositions

The methods described herein can also include the administrations of pharmaceutically acceptable compositions that include the therapeutic, or a pharmaceutically acceptable salt, solvate, or prodrug thereof. When employed as pharmaceuticals, any of the present compounds can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical, parenteral, intravenous, intra-arterial, subcutaneous, intramuscular, intracranial, intraorbital, ophthalmic, intraventricular, intracapsular, intraspinal, intracisternal, intraperitoneal, intranasal, aerosol, by suppositories, or oral administration.

This invention also includes pharmaceutical compositions which can contain one or more pharmaceutically acceptable carriers. In making the pharmaceutical compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, powders, lozenges, sachets, cachets, elixirs, suspensions, emulsions, solutions, syrups, and soft and hard gelatin capsules. As is known in the art, the type of diluent can vary depending upon the intended route of administration. The resulting compositions can include additional agents, such as preservatives.

The therapeutic agents of the invention can be administered alone, or in a mixture, in the presence of a pharmaceutically acceptable excipient or carrier. The excipient or carrier is selected on the basis of the mode and route of administration. In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

Examples of suitable excipients are lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents.

The methods described herein can include the administration of a therapeutic, or prodrugs or pharmaceutical compositions thereof, or other therapeutic agents. Exemplary therapeutics include those that bind the HER2 receptor and prevent HER2 dimerization with other receptors of the EGFR family, that Inhibit HER2 protein-protein interactions (PPIs), and prevent HER2 dimerization-mediated cell signaling (including, eg., SFTI-G5).

The pharmaceutical compositions can be formulated so as to provide immediate, extended, or delayed release of the active ingredient after administration to the patient by employing procedures known in the art.

The compositions can be formulated in a unit dosage form, each dosage containing, e.g., 0.1-500 mg of the active ingredient. For example, the dosages can contain from about 0.1 mg to about 50 mg, from about 0.1 mg to about 40 mg, from about 0.1 mg to about 20 mg, from about 0.1 mg to about 10 mg, from about 0.2 mg to about 20 mg, from about 0.3 mg to about 15 mg, from about 0.4 mg to about 10 mg, from about 0.5 mg to about 1 mg; from about 0.5 mg to about 100 mg, from about 0.5 mg to about 50 mg, from about 0.5 mg to about 30 mg, from about 0.5 mg to about 20 mg, from about 0.5 mg to about 10 mg, from about 0.5 mg to about 5 mg; from about 1 mg from to about 50 mg, from about 1 mg to about 30 mg, from about 1 mg to about 20 mg, from about 1 mg to about 10 mg, from about 1 mg to about 5 mg; from about 5 mg to about 50 mg, from about 5 mg to about 20 mg, from about 5 mg to about 10 mg; from about 10 mg to about 100 mg, from about 20 mg to about 200 mg, from about 30 mg to about 150 mg, from about 40 mg to about 100 mg, from about 50 mg to about 100 mg of the active ingredient, from about 50 mg to about 300 mg, from about 50 mg to about 250 mg, from about 100 mg to about 300 mg, or, from about 100 mg to about 250 mg of the active ingredient. For preparing solid compositions such as tablets, the principal active ingredient is mixed with one or more pharmaceutical excipients to form a solid bulk formulation composition containing a homogeneous mixture of a compound of the present invention. When referring to these bulk formulation compositions as homogeneous, the active ingredient is typically dispersed evenly throughout the composition so that the composition can be readily subdivided into equally effective unit dosage forms such as tablets and capsules. This solid bulk formulation is then subdivided into unit dosage forms of the type described above containing from, for example, 0.1 to about 500 mg of the active ingredient of the present invention.

Compositions for Oral Administration

The pharmaceutical compositions contemplated by the invention include those formulated for oral administration (“oral dosage forms”). Oral dosage forms can be, for example, in the form of tablets, capsules, a liquid solution or suspension, a powder, or liquid or solid crystals, which contain the active ingredient(s) in a mixture with non-toxic pharmaceutically acceptable excipients. These excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

Formulations for oral administration may also be presented as chewable tablets, as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders, granulates, and pellets may be prepared using the ingredients mentioned above under tablets and capsules in a conventional manner using, e.g., a mixer, a fluid bed apparatus or a spray drying equipment.

Controlled release compositions for oral use may be constructed to release the active drug by controlling the dissolution and/or the diffusion of the active drug substance. Any of a number of strategies can be pursued in order to obtain controlled release and the targeted plasma concentration vs time profile. In one example, controlled release is obtained by appropriate selection of various formulation parameters and ingredients, including, e.g., various types of controlled release compositions and coatings. Thus, the drug is formulated with appropriate excipients into a pharmaceutical composition that, upon administration, releases the drug in a controlled manner. Examples include single or multiple unit tablet or capsule compositions, oil solutions, suspensions, emulsions, microcapsules, microspheres, nanoparticles, patches, and liposomes. In certain embodiments, compositions include biodegradable, pH, and/or temperature-sensitive polymer coatings.

Dissolution or diffusion controlled release can be achieved by appropriate coating of a tablet, capsule, pellet, or granulate formulation of compounds, or by incorporating the compound into an appropriate matrix. A controlled release coating may include one or more of the coating substances mentioned above and/or, e.g., shellac, beeswax, glycowax, castor wax, carnauba wax, stearyl alcohol, glyceryl monostearate, glyceryl distearate, glycerol palmitostearate, ethylcellulose, acrylic resins, dl-polylactic acid, cellulose acetate butyrate, polyvinyl chloride, polyvinyl acetate, vinyl pyrrolidone, polyethylene, polymethacrylate, methylmethacrylate, 2-hydroxymethacrylate, methacrylate hydrogels, 1,3 butylene glycol, ethylene glycol methacrylate, and/or polyethylene glycols. In a controlled release matrix formulation, the matrix material may also include, e.g., hydrated methylcellulose, carnauba wax and stearyl alcohol, carbopol 934, silicone, glyceryl tristearate, methyl acrylate-methyl methacrylate, polyvinyl chloride, polyethylene, and/or halogenated fluorocarbon.

The liquid forms in which the compounds and compositions of the present invention can be incorporated for administration orally include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions suitable for oral mucosal administration (e.g., buccal or sublingual administration) include tablets, lozenges, and pastilles, where the active ingredient is formulated with a carrier, such as sugar, acacia, tragacanth, or gelatin and glycerine.

Coatings

The pharmaceutical compositions formulated for oral delivery, such as tablets or capsules of the present invention can be coated or otherwise compounded to provide a dosage form affording the advantage of delayed or extended release. The coating may be adapted to release the active drug substance in a predetermined pattern (e.g., in order to achieve a controlled release formulation) or it may be adapted not to release the active drug substance until after passage of the stomach, e.g., by use of an enteric coating (e.g., polymers that are pH-sensitive (“pH controlled release”), polymers with a slow or pH-dependent rate of swelling, dissolution or erosion (“time-controlled release”), polymers that are degraded by enzymes (“enzyme-controlled release” or “biodegradable release”) and polymers that form firm layers that are destroyed by an increase in pressure (“pressure-controlled release”)). Exemplary enteric coatings that can be used in the pharmaceutical compositions described herein include sugar coatings, film coatings (e.g., based on hydroxypropyl methylcellulose, methylcellulose, methyl hydroxyethylcellulose, hydroxypropylcellulose, carboxymethylcellulose, acrylate copolymers, polyethylene glycols and/or polyvinylpyrrolidone), or coatings based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose. Furthermore, a time delay material such as, for example, glyceryl monostearate or glyceryl distearate, may be employed.

For example, the tablet or capsule can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release.

When an enteric coating is used, desirably, a substantial amount of the drug is released in the lower gastrointestinal tract.

In addition to coatings that effect delayed or extended release, the solid tablet compositions may include a coating adapted to protect the composition from unwanted chemical changes (e.g., chemical degradation prior to the release of the active drug substance). The coating may be applied on the solid dosage form in a similar manner as that described in Encyclopedia of Pharmaceutical Technology, vols. 5 and 6, Eds. Swarbrick and Boyland, 2000.

Parenteral Administration

Within the scope of the present invention are also parenteral depot systems from biodegradable polymers. These systems are injected or implanted into the muscle or subcutaneous tissue and release the incorporated drug over extended periods of time, ranging from several days to several months. Both the characteristics of the polymer and the structure of the device can control the release kinetics which can be either continuous or pulsatile. Polymer-based parenteral depot systems can be classified as implants or microparticles. The former are cylindrical devices injected into the subcutaneous tissue whereas the latter are defined as spherical particles in the range of 10-100 μm. Extrusion, compression or injection molding are used to manufacture implants whereas for microparticles, the phase separation method, the spray-drying technique and the water-in-oil-in-water emulsion techniques are frequently employed. The most commonly used biodegradable polymers to form microparticles are polyesters from lactic and/or glycolic acid, e.g. poly(glycolic acid) and poly(L-lactic acid) (PLG/PLA microspheres). Of particular interest are in situ forming depot systems, such as thermoplastic pastes and gelling systems formed by solidification, by cooling, or due to the sol-gel transition, cross-linking systems and organogels formed by amphiphilic lipids. Examples of thermosensitive polymers used in the aforementioned systems include, N-isopropylacrylamide, poloxamers (ethylene oxide and propylene oxide block copolymers, such as poloxamer 188 and 407), poly(N-vinyl caprolactam), poly(siloethylene glycol), polyphosphazenes derivatives and PLGA-PEG-PLGA.

Mucosal Drug Delivery

Mucosal drug delivery (e.g., drug delivery via the mucosal linings of the nasal, rectal, vaginal, ocular, or oral cavities) can also be used in the methods described herein. Methods for oral mucosal drug delivery include sublingual administration (via mucosal membranes lining the floor of the mouth), buccal administration (via mucosal membranes lining the cheeks), and local delivery (Harris et al., Journal of Pharmaceutical Sciences, 81(1): 1-10, 1992).

Oral transmucosal absorption is generally rapid because of the rich vascular supply to the mucosa and allows for a rapid rise in blood concentrations of the therapeutic.

For buccal administration, the compositions may take the form of, e.g., tablets, lozenges, etc. formulated in a conventional manner. Permeation enhancers can also be used in buccal drug delivery. Exemplary enhancers include 23-lauryl ether, aprotinin, azone, benzalkonium chloride, cetylpyridinium chloride, cetyltrimethylammonium bromide, cyclodextrin, dextran sulfate, lauric acid, lysophosphatidylcholine, methol, methoxysalicylate, methyloleate, oleic acid, phosphatidylcholine, polyoxyethylene, polysorbate 80, sodium EDTA, sodium glycholate, sodium glycodeoxycholate, sodium lauryl sulfate, sodium salicylate, sodium taurocholate, sodium taurodeoxycholate, sulfoxides, and alkyl glycosides. Bioadhesive polymers have extensively been employed in buccal drug delivery systems and include cyanoacrylate, polyacrylic acid, hydroxypropyl methylcellulose, and poly methacrylate polymers, as well as hyaluronic acid and chitosan.

Liquid drug formulations (e.g., suitable for use with nebulizers and liquid spray devices and electrohydrodynamic (EHD) aerosol devices) can also be used. Other methods of formulating liquid drug solutions or suspension suitable for use in aerosol devices are known to those of skill in the art (see, e.g., Biesalski, U.S. Pat. No. 5,112,598, and Biesalski, U.S. Pat. No. 5,556,611).

Formulations for sublingual administration can also be used, including powders and aerosol formulations. Exemplary formulations include rapidly disintegrating tablets and liquid-filled soft gelatin capsules.

Dosing Regimes

The present methods for treating cancers are carried out by administering a therapeutic for a time and in an amount sufficient to result in decreased cancer progression, ceased cancer progression, or partial or complete cancer regression

The amount and frequency of administration of the compositions can vary depending on, for example, what is being administered, the state of the patient, and the manner of administration. In therapeutic applications, compositions can be administered to a patient suffering from cancer in an amount sufficient to relieve or least partially relieve the symptoms of the cancer and its complications. The dosage is likely to depend on such variables as the type and extent of progression of the cancer, the severity of the cancer, the age, weight and general condition of the particular patient, the relative biological efficacy of the composition selected, formulation of the excipient, the route of administration, and the judgment of the attending clinician. Effective doses can be extrapolated from dose-response curves derived from in vitro or animal model test system. An effective dose is a dose that produces a desirable clinical outcome by, for example, improving a sign or symptom of the cancer or slowing its progression.

The amount of therapeutic per dose can vary. For example, a subject can receive from about 0.1 μg/kg to about 10,000 μg/kg. Generally, the therapeutic is administered in an amount such that the peak plasma concentration ranges from 150 nM-250 μM.

Exemplary dosage amounts can fall between 0.1-5000 μg/kg, 100-1500 μg/kg, 100-350 μg/kg, 340-750 μg/kg, or 750-1000 μg/kg. Exemplary dosages can 0.25, 0.5, 0.75, 1°, or 2 mg/kg. In another embodiment, the administered dosage can range from 0.05-5 mmol of therapeutic (e.g., 0.089-3.9 mmol) or 0.1-50 μmol of therapeutic (e.g., 0.1-25 μmol or 0.4-20 μmol).

The plasma concentration of therapeutic can also be measured according to methods known in the art. Exemplary peak plasma concentrations of therapeutic can range from 0.05-10 μM, 0.1-10 μM, 0.1-5.0 μM, or 0.1-1 μM. Alternatively, the average plasma levels of therapeutic can range from 400-1200 μM (e.g., between 500-1000 μM) or between 50-250 μM (e.g., between 40-200 μM). In some embodiments where sustained release of the drug is desirable, the peak plasma concentrations (e.g., of therapeutic) may be maintained for 6-14 hours, e.g., for 6-12 or 6-10 hours. In other embodiments where immediate release of the drug is desirable, the peak plasma concentration (e.g., of therapeutic) may be maintained for, e.g., 30 minutes.

The frequency of treatment may also vary. The subject can be treated one or more times per day with therapeutic (e.g., once, twice, three, four or more times) or every so-many hours (e.g., about every 2, 4, 6, 8, 12, or 24 hours). Preferably, the pharmaceutical composition is administered 1 or 2 times per 24 hours. The time course of treatment may be of varying duration, e.g., for two, three, four, five, six, seven, eight, nine, ten or more days. For example, the treatment can be twice a day for three days, twice a day for seven days, twice a day for ten days. Treatment cycles can be repeated at intervals, for example weekly, bimonthly or monthly, which are separated by periods in which no treatment is given. The treatment can be a single treatment or can last as long as the life span of the subject (e.g., many years).

Kits

Any of the pharmaceutical compositions of the invention described herein can be used together with a set of instructions, i.e., to form a kit. The kit may include instructions for use of the pharmaceutical compositions as a therapy as described herein. For example, the instructions may provide dosing and therapeutic regimes for use of the compounds of the invention to reduce symptoms and/or underlying cause of the cancer.

The invention illustratively disclosed herein suitably may explicitly be practiced in the absence of any element which is not specifically disclosed herein. While various embodiments of the present invention have been described in detail, it is apparent that various modifications and alterations of those embodiments will occur to and be readily apparent those skilled in the art. However, it is to be expressly understood that such modifications and alterations are within the scope and spirit of the present invention, as set forth in the appended claims. Further, the invention(s) described herein is capable of other embodiments and of being practiced or of being carried out in various other related ways. In addition, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items while only the terms “consisting of” and “consisting only of” are to be construed in the limitative sense.

Wherefore, I/we claim:

1. A method of treating cancer in a mammalian patient comprising:
administering a pharmaceutical composition to the patient including a pharmacologically effective amount of a first therapeutic,
wherein the first therapeutic comprises one or more HER2-targeted peptide functional groups grafted onto a cyclic scaffold, or a pharmaceutically acceptable salt, solvate, ester, amide, clathrate, stereoisomer, enantiomer, prodrug or analogs thereof.

2. The method of claim 1, wherein one or more of the functional groups of the peptide bind to region IV of an extracellular domain of HER2.

3. The method of claim 1, wherein the scaffold is one of a sunflower trypsin inhibitor and a theta-defensins.

4. The method of claim 1 wherein the one or more HER2-targeted peptide functional groups grafted onto a cyclic scaffold contains at least one 3-amino 3(1-napthyl) propionic acid bound to the scaffold.

5. The method of claim 4 wherein the one or more HER2-targeted peptide functional groups grafted onto a cyclic scaffold contains at least two 3-amino 3(1-napthyl) propionic acids bound to the scaffold.

6. The method of claim 5 wherein two of the at least two 3-amino 3(1-napthyl) propionic acids are of the same chirality.

7. The method of claim 5 wherein two of the at least two 3-amino 3(1-napthyl) propionic acids are bonded to each other by between 2 and 7 amino acids.

8. The method of claim 1, wherein a sequence of the grafted peptide is one of
Cyclo(C(S-X)RDR(S-X)CFDSIDF);
Cyclo(C(R-X)RPpR(R-X)CFF);
Cyclo(C(R-X)RRPR(R-X)CF);
Cyclo(C(S-X)RPGR(S-X)CFDSIPPDF);
Cyclo(C(S-X)RIPPR(S-X)CFPDDF);
Cyclo(C(R-X)RIPPR(R-X)CFPDDF);
Cyclo(C(S-X)RIHyPHyPR(S-X)CFPDDF);
(C-(S-X)RI-HyPHyP(S-X)CFPDDF);
Cyclo(C(S-X)RIHyPHyPR(S-X)CFpddf);
Cyclo(C(S-X)RI-DBF-R(S-X)CFPDDF);
Cyclo(C(S-X)RI-HyDBF-R(S-X)CFPDDF);
Cyclo(C(S-X)-K-RIPPR(S-X)CFPDDF);
where “R-X” and “S-X” indicate the chirality at a beta amino acid; “X” is a beta amino acid 3-amino 3(1-napthyl) propionic acid; “K” is lysine amino acid; single letter code is used for amino acid representation, capital letters refer to L amino acid, lower case letters refers to D amino acid, “Hy” before an amino acid indicates a hydroxyl form of the following amino acid, a disulfide bond between cysteines is shown by underline, “Cyclo” indicates a cyclic compound scaffold; “DBF” is dibenzofuran, and “HyDBF” is hydroxyl dibenzofuran.

9. The method of claim 8, where one of a side chain of K is attached with a fluorescent labels and K is modified with azide group and attached to a fluorescent label.

10. The method of claim 1 wherein the pharmaceutical composition further comprises a second therapeutic distinct from the first therapeutic.

11. The method of claim 10 wherein the second therapeutic includes one or more chemotherapeutic agents.

12. The method of claim 10 wherein the second therapeutic includes one or more of cisplatin, paclitaxel, and docetaxel, or a pharmaceutically acceptable salt, solvate, ester, amide, clathrate, stereoisomer, enantiomer, prodrug or analogs thereof.

13. The method of claim 10 wherein the second therapeutic includes one or more tyrosine kinase inhibitors.

14. The method of claim 10 wherein the second therapeutic includes one of erlotinib, gefitinib, and lapatinib, or a pharmaceutically acceptable salt, solvate, ester, amide, clathrate, stereoisomer, enantiomer, prodrug or analogs thereof.

15. The method of claim 11 wherein the pharmaceutical composition further comprises a third therapeutic distinct from the first therapeutic and the second therapeutic.

16. The method of claim 15 wherein the third therapeutic includes one or more tyrosine kinase inhibitors.

17. The method of claim 1 wherein the cancer is one of a lung, a breast, an ovary, a bladder, a salivary gland, a pancreas, and an endometrium cancer.

18. The method of claim 1 wherein the cancer is HER2 positive.

19. The method of claim 8, wherein
the peptide sequence one of freely changes conformations, is locked into beta conformation through incorporation of 1,4 substituted triazole, is locked into beta conformation through substitution of Pro-Pro with dibenzofuran,
the mammal is one of Homo sapiens and Canis lupus,
the cancer is HER2+ and the HER2 is one of wild-type a mutated variant, the administration route is one of oral, intravenous, or inhalation, the first therapeutic is administered with pharmaceutically acceptable excipients.

20. A method of visualizing HER2+ cancers comprising:
using a peptide consisting of a sequence Cyclo(C-(S-X)-KRI-PPR-(S-X)-CFPDDF),
wherein
“S-X” indicates a chirality at a beta amino acid,
“X” is a beta amino acid 3-amino 3(1-napthyl) propionic acid;
“K” is lysine amino acid, single letter code is used for amino acid representation,
capital letters refer to L amino acid and lower case letters refers to D amino acid,
“Hy” before an amino acid indicates a hydroxyl form of the following amino acid,
a disulfide bond between cysteines is shown by underline,
“Cyclo” indicates a cyclic compound scaffold, and
X is amino naphthyl propionic acid (Anapa).


SEQUENCE LISTING (12 sequences)

SEQ ID NO: 1 - protein, 13 aa, Helianthus annuus
  misc_feature (2)..(2): Xaa is 3-amino 3(1-napthyl) propionic acid
  misc_feature (6)..(6): Xaa is 3-amino 3(1-napthyl) propionic acid
CXRDRXCFDSIDF

SEQ ID NO: 2 - protein, 10 aa, Helianthus annuus
  misc_feature (2)..(2): Xaa is 3-amino 3(1-napthyl) propionic acid
  misc_feature (7)..(7): Xaa is 3-amino 3(1-napthyl) propionic acid
CXRPPRXCFF

SEQ ID NO: 3 - protein, 9 aa, Helianthus annuus
  misc_feature (2)..(2): Xaa is 3-amino 3(1-napthyl) propionic acid
  misc_feature (7)..(7): Xaa is 3-amino 3(1-napthyl) propionic acid
CXRRPRXCF

SEQ ID NO: 4 - protein, 16 aa, Helianthus annuus
  misc_feature (2)..(2): Xaa is 3-amino 3(1-napthyl) propionic acid
  misc_feature (7)..(7): Xaa is 3-amino 3(1-napthyl) propionic acid
CXRPGRXCFDSIPPDF

SEQ ID NO: 5 - protein, 14 aa, Helianthus annuus
  misc_feature (2)..(2): Xaa is 3-amino 3(1-napthyl) propionic acid
  misc_feature (8)..(8): Xaa is 3-amino 3(1-napthyl) propionic acid
CXRIPPRXCFPDDF

SEQ ID NO: 6 - protein, 14 aa, Helianthus annuus
  misc_feature (2)..(2): Xaa is 3-amino 3(1-napthyl) propionic acid
  misc_feature (8)..(8): Xaa is 3-amino 3(1-napthyl) propionic acid
CXRIPPRXCFPDDF

SEQ ID NO: 7 - protein, 16 aa, Helianthus annuus
  misc_feature (2)..(2): Xaa is 3-amino 3(1-napthyl) propionic acid
  misc_feature (10)..(10): Xaa is 3-amino 3(1-napthyl) propionic acid
CXRIPHYPRXCFPDDF

SEQ ID NO: 8 - protein, 15 aa, Helianthus annuus
  misc_feature (2)..(2): Xaa is 3-amino 3(1-napthyl) propionic acid
  misc_feature (9)..(9): Xaa is 3-amino 3(1-napthyl) propionic acid
CXRIPHYPXCFPDDF

SEQ ID NO: 9 - protein, 16 aa, Helianthus annuus
  misc_feature (2)..(2): Xaa is 3-amino 3(1-napthyl) propionic acid
  misc_feature (10)..(10): Xaa is 3-amino 3(1-napthyl) propionic acid
CXRIHYPPRXCFPDDF

SEQ ID NO: 10 - protein, 15 aa, Helianthus annuus
  misc_feature (2)..(2): Xaa is 3-amino 3(1-napthyl) propionic acid
  misc_feature (9)..(9): Xaa is 3-amino 3(1-napthyl) propionic acid
CXRIDBFRXCFPDDF

SEQ ID NO: 11 - protein, 15 aa, Helianthus annuus
  misc_feature (2)..(2): Xaa is 3-amino 3(1-napthyl) propionic acid
  misc_feature (9)..(9): Xaa is 3-amino 3(1-napthyl) propionic acid
CXRIDBFRXCFPDDF

SEQ ID NO: 12 - protein, 15 aa, Helianthus annuus
  misc_feature (2)..(2): Xaa is 3-amino 3(1-napthyl) propionic acid
  misc_feature (9)..(9): Xaa is 3-amino 3(1-napthyl) propionic acid
CXKRIPPRXCFPDDF